(19) United States
(12) Patent Application Publication
(10) Pub. No.: US 2018/0292386 A1
(43) Pub. Date: Oct. 11, 2018

(54) METHOD FOR PREDICTING A DRUG RESPONSE BASED ON THE ATTRACTOR DYNAMICS OF THE NETWORK OF A CANCER-CELL AND DEVICE FOR THE SAME

(71) Applicant: KOREA ADVANCED INSTITUTE OF SCIENCE AND TECHNOLOGY, Daejeon (KR)

(72) Inventors: Kwang-Hyun CHO, Daejeon (KR); Min-Soo CHOI, Seoul (KR)

(21) Appl. No.: 15/715,716

(22) Filed: Sep. 26, 2017

(30) Foreign Application Priority Data
Apr. 5, 2017 (KR) 10-2017-0044192

(51) Int. Cl. G01N 33/50; G01N 33/574

(52) U.S. Cl. CPC G01N 33/5008; G06F 19/12; G01N 33/574; G01N 33/5091

(57) ABSTRACT

A drug efficiency calculation method comprises generating information regarding a perturbation state transition diagram which is a state transition diagram of a specific perturbation network generated by applying a perturbation corresponding to a specific drug to a specific network generated by mapping the gene mutation information of a cancer-cell to a nominal network, and calculating a score regarding the efficiency of the drug on the basis of the size of one or more basins of the perturbation state transition diagram.

### CROSS-REFERENCE TO RELATED APPLICATION

This application claims priority to Korean Patent Application No. 10-2017-0044192 filed on Apr. 5, 2017, and all the benefits accruing therefrom under 35 U.S.C. § 119, the contents of which are incorporated by reference in their entirety.

### ACKNOWLEDGEMENT

This work was supported by the National Research Foundation of Korea (NRF) grants funded by the Korea Government, the Ministry of Science and ICT (2017R1A2A1A17069642 and 2015M3A9A7067220).

### BACKGROUND

The present invention relates to an information processing technique using a computer and especially to technique for calculating an evaluation value regarding effect of a drug on a normal cell and a cancer-cell using information regarding the cancer-cell and the drug.

When a bio-molecular network is regarded as one system, a state of the system may be modeled as a Boolean network expressed a set of the state (“ON (1)” or “OFF (0)”) of all molecules comprising the network. For example the state-space size (the number of possible initial conditions) of the bio-molecular network having n molecules is 2ⁿ and a state transition graph may be obtained by analyzing state transition of all possible initial conditions.

The state transition occurs in the state of the system according to time due to dynamics of inside the system and a finally reached state is called an attractor. Here, a set of all initial conditions which converge on a same attractor is called a basin of attraction. An attractor is divided into a point attractor of a fixed state and a cyclic attractor whose state is periodically repeated.

There is a Korean patent application Ser. No. 2012-0098296 as a technique analyzing characteristics of the above-stated Boolean network. Technical contents written in the Korean patent application Ser. No. 2012-0098296 may be included in the present invention as a reference.

Meanwhile, dynamic characteristics of a normal cell and a cancer-cell may be modeled by using the bio-molecular network. Here, a state which the normal cell and the cancer-cell obtained conclusively may be associated with the point attractor or the cyclic attractor and each point attractor or the cyclic attractor represents a particular phenotype of the normal cell and the cancer-cell such as cell proliferation, cell cycle arrest or cell-death.

For example, nodes composing bio-molecular network of the normal cell and the cancer-cell may correspond to a molecule such as a gene or a protein. Also, when a specific drug is administered to a normal cell and a cancer-cell, in a case that a gene or a protein which is affected by the drug exists, the bio-molecular network may be changed using a perturbation by which the value of the node is restricted to a specific range, wherein the node correspond to the gene or the protein which is effected by the drug. By analyzing dynamic characteristics of a transformed bio-molecular network, an effect of a specific drug on a normal cell and a cancer-cell may be analyzed through a computer simulation.

The above-mentioned content is merely a description of a background technique of the present invention and not all of the description is necessarily regarded as prior art to the present invention.

### SUMMARY

The present invention provides a method calculating an effect of a particular drug on a phenotype of a normal cell and a cancer-cell through a computer simulation.

A method of analysis according to one aspect of the present invention uses methods for identifying a attractor and a basin through a state transition analysis and investigating dynamic characteristics of a bio-molecular network, by using a Boolean mathematical model of a bio-molecular network.

Here, the value of each node in the bio-molecular network may be 0 (zero) or 1. Particularly, with a probability of x %, the value of selected one node among the all nodes in the bio-molecular network may be 0 (zero), and with a probability of (100−x) %, the value of the selected one node may be 0 (zero) or 1 which is determined by the logic ruling the bio-molecular network. The selected one node may be a target node whose activation is inhibited by a drug or a drug-combination.

A method of analysis according to one aspect of the present invention acquires a state transition graph changed through a perturbation analysis simulating a drug effect in result and analyses how a targeting drug induces a different phenotype of a cell by analyzing an attractor.

According to an analysis method according to one aspect of the present invention, only the mutation information which can affect the function of a gene may be selected among the gene mutation information of a cancer-cell. And according to the analysis method, according to the functional type of the selected mutation information, the state of a node of the bio-molecular network which corresponding to the gene is left in an activated state or an inactivated state. Here, a particular node is always left in an activated state or an inactivated state in an embodiment, or in another embodiment, the value of the particular node may be 0 (zero) or 1 (one) with a predetermined probability.

Then, in the analysis method, a state attractor-based perturbation analysis simulating an action mechanism of a drug on a specific network specialized in a cancer-cell constructed through this process may be performed. Consequently, it is possible to identify a specific phenotype that a cancer-cell obtains as a result by an action of drug and a ratio at which the phenotype may be generated. Also, the analysis method includes a method for analyzing a dynamic characteristic of network changed by a drug. With this, not only the prediction of a drug reaction but also an action mechanism inducing drug resistance may be investigated and an optimal drug target may be presented.

A method of analysis according to one aspect of the present invention includes the process predicting a drug efficacy (based on the drug efficacy score) regarding a cancer-cell and a synergy effect (based on the drug synergy score) through the analyzing method based on the attractor dynamics. Also, for an efficacy analysis of a drug regarding the cancer-cell, the method of analysis may include a calculated step by comparing the difference of a response phenotype score of before/after a drug treatment through a perturbation analysis simulating a drug effect. Further, the method of analysis may perform the step calculating the synergy effect through the difference in the ratio of a phenotype leading to cell-death of the cancer-cell between a predicted reaction when drugs are combined and a real reaction.

According to one aspect of the present invention, a method for generating a first specific network by mapping the gene mutation information of a first cancer-cell to a nominal network may be provided. The method may include a step acquiring the nominal network; a step obtaining the gene mutation information of the first cancer-cell; a step finding a first node corresponding to a nonsense (because the synthesis of a protein is stopped by change of a base sequence, an incomplete protein is synthesized) mutated gene or a HOMDEL (homozygous deletion/a replication region is omitted/HOMDEL corresponds to a deep loss) copy-number-variated gene of the first cancer-cell in the nominal network and changing the nominal network such that the first node is always in an inactive state; a step finding a second node corresponding to a gene whose functional impact score (FIS/based on the base sequence information, quantify the degree to which the mutation of the gene is expected to affect the function and group them into neutral/low/medium/high for example/if FIS is medium or high, it may be predicted that a gene does an abnormal function because of the corresponding mutation) is greater than a pre-determined first value among missense mutated gene of the first cancer-cell in the nominal network, if the missense (in some cases, an abnormal protein may be synthesized by change of the base sequence) mutated gene is an Oncogene, changing the nominal network such that the second node is always in an active state, or if the missense mutated gene is a Tumor suppressor, changing the nominal network such that the second node is always in an inactive state; a step finding a third node corresponding to a gene whose z score (it is calculated by using the distribution of mRNA expression of a gene whose copy number is normal (diploid)) of mRNA expression is smaller than a pre-determined second value (z score <−2) among LOSS (the copy number is relatively lower than that of a normal gene) copy-number-variated genes of the first cancer-cell, and changing the nominal network such that the third node is always in an inactive state; and a step finding a fourth node corresponding to a gene whose z score of mRNA expression is greater than a pre-determined third value (z score >2) among GAIN (the copy number is relatively higher than that of the normal gene) copy-number-variated genes or AMP (the copy number is higher than that of GAIN/it corresponds to high-level GAIN) copy-number-variated genes of the first cancer-cell, and changing the nominal network such that the fourth node is always in an active state. The each step may be performed by a computing device. The each step may be performed in the computing device by a program, and according to one aspect of the present invention, a non-transitory recording medium which may be readable by computer in which the program is stored may be provided.

A drug efficiency calculation method provided according to one aspect of the present invention, includes the steps of: generating a first specific network by mapping the gene mutation information of a first cancer-cell to a nominal network; generating a first specific perturbation network by applying a first perturbation corresponding to a first drug to the first specific network; generating information regarding a first perturbation state transition diagram, which is a state transition diagram of the first specific perturbation network; and calculating a score regarding the efficiency of the first drug on the basis of the size of one or more basins of the first perturbation state transition diagram.

In this specification, the term ‘perturbation network’ and ‘perturbation state’ may be rewritten with a term ‘perturbated network’ and ‘perturbated state’, respectively.

Here, the first drug may be a combination of two or more different drugs.

Here, further the drug efficiency calculation method may include the steps of: generating a second specific perturbation network by applying a second perturbation corresponding to a second drug or a second drug-combination to the first specific network; generating information regarding a second perturbation state transition diagram, which is a state transition diagram of the second specific perturbation network; generating a third specific perturbation network by applying a third perturbation corresponding to a combination of the first drug and the second drug to the first specific network; and generating information regarding a third perturbation state transition diagram, which is a state transition diagram of the third specific perturbation network,

Here, the score regarding the efficiency may include a synergy score representing a synergy effect according to the combination of the first drug and the second drug.

Here, a method for calculating the synergy score may include the steps of; calculating a first D-ratio (D_{A}), which is a probability with which the first cancer-cell is led to the cell-death state, on the basis of the size of a basin representing the cell-death that can be obtained respectively from the information regarding the first perturbation state transition diagram; calculating a second D-ratio (D_{B}), which is a probability with which the first cancer-cell is led the cell-death state, on the basis of the size of a basin representing the cell-death that can be obtained respectively from the information regarding the second perturbation state transition diagram; calculating a third D-ratio (D_{AB}), which is a probability with which the first cancel-cell is led the cell-death state, on the basis of the size of a basin representing the cell-death that can be obtained respectively from the information regarding the third perturbation state transition diagram; and calculating the synergy score S score using the first D-ratio, the second D-ratio, and the third D-ratio.

Here, the synergy score is calculated using the following FORMULA 4.

S score=D_{AB}−{1−(1−D_{A})(1−D_{B})} [FORMULA 4]

Here, the score regarding the efficiency may include an efficacy score D score which is calculated by using \textcircled{1} a first R score R_{before} calculated by applying a first proliferation probability P_{P}_{_}_{before}, a first cycle arrest probability P_{A}_{_}_{before}, and a first death probability P_{D}_{_}_{before}, which are respectively proportional to the size of basins representing the cell proliferation, the cell cycle arrest and the cell-death of a first state transition diagram which is the state transition diagram of the first specific network, respectively into P_{P}, P_{A}, P_{D} of the following FORMULA 1, and \textcircled{2} a second R score R_{after} calculated by applying a second proliferation probability P_{P}_{_}_{after}, a second cycle arrest probability P_{A}_{_}_{after}, and a second death probability P_{D}_{_}_{after}, which are respectively proportional to the size of basins representing the cell proliferation, the cell cycle arrest and the cell-death of the first perturbation state transition diagram, respectively into P_{P}, P_{A}, P_{D} of the following FORMULA 1.

R score=W_{P}*P_{P}+W_{A}*P_{A}+W_{D}*P_{D} [FORMULA 1]

W_{P}, W_{A}, and W_{D} are a pre-determined constant, respectively

Here, the efficacy score (D score) may be calculated using the following FORMULA 2.

D score=(R_{after}−R_{before})/(Rₘₐₓ−R_{before}) [FORMULA 2]

Here, the Rₘₐₓ is the maximum value which the R score of FORMULA 1 may have.

Here, the first drug or the second drug may be a combination of two or more different drugs.

Here, the generating the first specific network by mapping the gene mutation information of the first cancer-cell to the nominal network includes the steps of: acquiring the nominal network; obtaining the gene mutation information of the first cancer-cell; finding a first node corresponding to a nonsense mutated gene or a HOMDEL copy-number-variated gene of the first cancer-cell in the nominal network and changing the nominal network such that the first node is always in an inactive state; finding a second node corresponding to a gene whose functional impact score is greater than a pre-determined first value among missense mutated genes of the first cancer-cell in the nominal network, if the missense mutated gene is an Oncogene, changing the nominal network such that the second node is always in an active state, or if the missense mutated gene is a Tumor suppressor, changing the nominal network such that the second node is always in an inactive state; finding a third node corresponding to a gene whose z score of mRNA expression is smaller than a pre-determined second value among LOSS copy-number-variated genes of the first cancer-cell, and changing the nominal network such that the third node is always in an inactive state; and finding a fourth node corresponding to a gene whose z score of mRNA expression is greater than a pre-determined third value among GAIN copy-number-variated genes or AMP copy-number-variated genes of the first cancer-cell, and changing the nominal network such that the fourth node is always in an active state.

A drug efficiency calculation method according to another aspect of the present invention, include the steps of: generating information regarding a perturbation state transition diagram which is a state transition diagram of a specific perturbation network generated by applying a perturbation corresponding to a specific drug to a specific network generated by mapping the gene mutation information of a cancer-cell to a nominal network; and calculating a score regarding the efficiency of the drug on the basis of the size of one or more basins of the perturbation state transition diagram.

A non-transitory computer readable recording medium according to the other aspect of the present invention includes program codes to be executed on a computing device to perform the steps of: generating a first specific network by mapping the gene mutation information of a first cancer-cell to a nominal network; generating a first specific perturbation network by applying a first perturbation corresponding to a first drug to the first specific network; generating information regarding a first perturbation state transition diagram, which is a state transition diagram of the first specific perturbation network; and calculating a score regarding an efficiency of the first drug on the basis of the size of one or more basins of the first perturbation state transition diagram.

A computing device according to the other aspect of the present invention includes at least one processor configured to perform operations comprising generating a first specific network by mapping the gene mutation information of a first cancer-cell to a nominal network; generating a first specific perturbation network by applying a first perturbation corresponding to a first drug to the first specific network; generating information regarding a first perturbation state transition diagram, which is a state transition diagram of the first specific perturbation network; and calculating a score regarding the efficiency of the first drug on the basis of the size of one or more basins of the first perturbation state transition diagram.

According to the present invention, it is possible to identify a specific phenotype that a cancer-cell obtains as a result by a drug and a ratio at which the phenotype may be generated. Also, it is possible to present not only prediction of drug response but also an investigation of an operation mechanism inducing drug resistance and a optimal drug target to overcome this.

According to the output of the drug reactivity calculation method and the drug efficiency calculation method of the present invention, the time required for selecting an optimum drug or drug-combination and selecting an optimum dosage rage can be dramatically reduced in a field of cancer treatment.

According to one aspect of the present invention, a drug efficiency calculation method is provided. The method comprises, generating, by the computing device, information regarding a first perturbation state transition diagram which is a state transition diagram of a first specific perturbation network, wherein, the first specific perturbation network is a network generated by applying a first perturbation corresponding to a first drug to a first specific network, the first specific network being a network generated by mapping gene mutation information of a first cancer-cell to a nominal network; and calculating, by the computing device, a score regarding the efficiency of the first drug on the basis of the size of one or more basins of the first perturbation state transition diagram.

The method may further comprises, generating, by the computing device, a second specific perturbation network by applying a second perturbation corresponding to a second drug or a second drug-combination to the first specific network; generating, by the computing device, information regarding a second perturbation state transition diagram which is a state transition diagram of the second specific perturbation network; generating, by the computing device, a third specific perturbation network by applying a third perturbation corresponding to a combination of the first drug and the second drug to the first specific network; and generating, by the computing device, information regarding a third perturbation state transition diagram which is a state transition diagram of the third specific perturbation network.

Particularly, the score regarding the efficiency may include a synergy score representing a synergy effect according to the combination of the first drug and the second drug.

Particularly, the synergy score is calculated by a method including, calculating, by the computing device, a first D-ratio (D_{A}), which is a probability with which the first cancer-cell is led to the cell-death state, on the basis of the size of a basin representing the cell-death that can be obtained respectively from the information regarding the first perturbation state transition diagram; calculating, by the computing device, a second D-ratio (D_{B}), which is a probability with which the first cancer-cell is led the cell-death state, on the basis of the size of a basin representing the cell-death that can be obtained respectively from the information regarding the second perturbation state transition diagram; calculating, by the computing device, a third D-ratio (D_{AB}), which is a probability with which the first cancel-cell is led the cell-death state, on the basis of the size of a basin representing the cell-death that can be obtained respectively from the information regarding the third perturbation state transition diagram; and calculating, by the computing device, the synergy score (S score) using the first D-ratio, the second D-ratio, and the third D-ratio.

Particularly, the score regarding the efficiency may include an efficacy score (D score) which is calculated by using \textcircled{1} a first R score (R_{before}) calculated by applying a first proliferation probability (P_{P}_{_}_{before}), a first cycle arrest probability (P_{A}_{_}_{before}), and a first death probability (P_{D}_{_}_{before}), which are respectively proportional to the size of basins representing the cell proliferation, the cell cycle arrest and the cell-death of a first state transition diagram which is the state transition diagram of the first specific network, respectively into P_{P}, P_{A}, P_{D} of FORMULA 1, and \textcircled{2} a second R score (R_{after}) calculated by applying a second proliferation probability (P_{P}_{_}_{after}), a second cycle arrest probability (P_{A}_{_}_{after}), and a second death probability (P_{D}_{_}_{after}), which are respectively proportional to the size of basins representing the cell proliferation, the cell cycle arrest and the cell-death of the first perturbation state transition diagram, respectively into P_{P}, P_{A}, P_{D} of FORMULA 1.

R score=W_{P}*P_{P}+W_{A}*P_{A}+W_{D}*P_{D} [FORMULA 1]

W_{P}, W_{A}, and W_{D} are a pre-determined constant, respectively

Particularly, the nominal network may be defined to comprise a plurality of nodes each of which corresponds to the function of the corresponding molecules in the cell and a plurality of links representing the directions of signals delivered between the plurality of nodes and the values of the signals, for each of the plurality of nodes, the value of the node is 0 (zero) or 1 (one), and for each of the links, when the value of a source node which is a source of the signal on the link of the plurality of nodes is changed, the value of a target node which is a target of the signal on the link is changed according to the value of the signal on the link, the value of one node of the plurality of nodes is controlled by the first perturbation, or the value of the signal on one link of the plurality of links is controlled by the first perturbation.

According to another aspect of the present invention, a drug efficiency calculation method can be provided. This method comprises, generating information regarding a perturbation state transition diagram which is a state transition diagram of a specific perturbation network generated by applying a perturbation corresponding to a drug to a specific network generated by mapping gene mutation information of a cancer-cell to a nominal network at a computing device; and calculating a score regarding the efficiency of the drug on the basis of the size of one or more basins of the perturbation state transition diagram at the computing device.

According to still another aspect of the present invention, a non-transitory computer readable recording medium having recorded thereon program codes can be provided. The program codes is designed to be executed on a computing device to perform the steps of: generating information regarding a first perturbation state transition diagram which is a state transition diagram of a first specific perturbation network, wherein, the first specific perturbation network is a network generated by applying a first perturbation corresponding to a first drug to a first specific network, the first specific network being a network generated by mapping the gene mutation information of a first cancer-cell to a nominal network; and calculating a score regarding an efficiency of the first drug on the basis of the size of one or more basins of the first perturbation state transition diagram.

According to still another aspect of the present invention, a computing device can be provided. This device comprises, at least one processor configured to perform operations comprising: generating a first specific network by mapping the gene mutation information of a first cancer-cell to a nominal network; generating a first specific perturbation network by applying a first perturbation corresponding to a first drug to the first specific network; generating information regarding a first perturbation state transition diagram, which is a state transition diagram of the first specific perturbation network; and calculating a score regarding the efficiency of the first drug on the basis of the size of one or more basins of the first perturbation state transition diagram.

According to still another aspect of the present invention, a method of calculating reactivity of a drug can be provided. This method comprises a drug-reactivity calculating process, which comprises: creating, by the computing device, information regarding a first nominal perturbation state transition diagram which is a state transition diagram of a first nominal perturbation network, wherein the first nominal perturbation network being is a network generated by applying a first perturbation to a nominal network modelled on interactions between molecules in a cell, the first perturbation corresponding to a dosage of a first drug; calculating, by the computing device, a side-effect score of the first drug according to the dosage of the first drug based on the sizes of basins of the first nominal perturbation state transition diagram; creating, by the computing device, information regarding a first perturbation state transition diagram which is a state transition diagram of a first specific perturbation network, wherein, the first specific perturbation network is a network created by applying the first perturbation to a first specific network, and the first specific network is a network created by mapping gene mutation information of a first cancer-cell to the nominal network; and calculating, by the computing device, an efficiency score of the first drug according to the dosage of the first drug based on the sizes of basins of the first perturbation state transition diagram.

Particularly, the nominal network may be defined to comprise a plurality of nodes each of which corresponds to the function of the corresponding molecules in the cell and a plurality of links representing the directions of signals delivered between the plurality of nodes and the values of the signals,

Particularly, for each of the plurality of nodes, the value of the node may be 0 (zero) or 1 (one),

Particularly, for each of the links, when the value of a source node of the plurality of nodes which is a source of the signal on the link is changed, the value of a target node which is a target of the signal on the link may be changed according to the value of the signal on the link, and

Particularly, the value of selected one node of the plurality of nodes may be controlled by the first perturbation, or the value of the signal on selected one link of the plurality of links may be controlled by the first perturbation.

Particularly, the computing device may be configured so that the value of the signal is set to only a first value or a second value, the first value being a value which makes the value of the target node increases when the value of the source node increases, and the second value being a value which makes the value of the target node decreases when the value of the source node increases.

Particularly, the computing device may be configured so that, (1) with a probability of x %, the value of selected one node among the all nodes in the bio-molecular network is set to 0 (zero), and (2) with a probability of (100−x) %, the value of the selected one node is set to 0 (zero) or 1 (one), which is determined by the logic ruling the bio-molecular network; and the computing device is configured so that the value of the signal output from the source node increases when the value of the source node increases.

Particularly, the strength of the first perturbation may be positively correlated with the dosage of the first drug, the method may further comprise a data acquisition process acquiring drug-reactivity data having pairs of [toxicity score, efficacy score] according to the dosage of the first drug by conducting the drug-reactivity calculating process a plurality of times with changing the strength of the first perturbation.

Particularly, the toxicity score may be calculated based on the sizes of the basins of a first nominal state transition diagram which is a state transition diagram of the nominal network, and the sizes of the basins of the first nominal perturbation-state transition diagram.

Particularly, the efficacy score may be calculated based on the sizes of the basins of a first state transition diagram which is a state transition diagram of the first specific network, and the sizes of the basins of the first perturbation state transition diagram.

Particularly, this method may further comprises an optimum dosage-range determination process calculating a dosage-range of the first drug which makes the toxicity score fall on a first predetermined score range, and the efficacy score fall on a second predetermined score range, or an optimum dosage-range determination process calculating a dosage-range of the first drug which makes the difference between the toxicity score and the efficacy score fall on a third predetermined score range.

### BRIEF DESCRIPTION OF THE DRAWINGS

Exemplary embodiments can be understood in more detail from the following description taken in conjunction with the accompanying drawings, in which:

FIG. 1 shows a diagram that a bio-molecular network as a network comprising links representing four nodes (x1˜x4) and correlation between them is modeled and a truth table which the values of the four nodes shown.

FIG. 2 shows a state transition diagram representing a total of sixteen states which the Boolean network shown in FIG. 1 may be have, and a diagram separately representing two point attractors and one cyclic attractor included in the state transition diagram shown.

FIG. 3 shows a bio-molecular network modeled for a real cancer-cell and a state transition diagram representing a total of 2̂16 states which the Boolean network shown may have.

FIG. 4 is a diagram for describing procedure of a drug prediction method based on an attractor dynamics analysis of a cancer-cell network provided according to an embodiment of the present invention.

FIG. 5 is a diagram for describing method for calculating the efficacy score of a drug according to an embodiment of the present invention.

FIG. 6 is a diagram for describing method calculating the synergy score of a drug-combination according to an embodiment of the present invention.

FIG. 7 is a flow chart for describing a drug efficiency calculation method according to an embodiment of the present invention.

FIG. 8 is a flow chart showing the method for calculating the synergy score shown in FIG. 7.

FIG. 9 is a flow chart showing the detailed method for generating the specific network by mapping the gene mutation information of the first cancer-cell to the nominal network shown in FIG. 7.

FIG. 10 shows an example of graphs representing the change of efficacy score and toxicity score according to the dosage of a first drug according to an embodiment of the present invention, and shows one way to decide an optimum dosage range of the first drug.

FIG. 11 shows an example of graphs representing the change of efficacy score and toxicity score according to the dosage of a second drug according to an embodiment of the present invention, and shows one way to decide an optimum dosage range of the second drug.

FIG. 12 shows an example of graphs representing the change of efficacy score and toxicity score according to the dosage of a first drug according to an embodiment of the present invention, and shows other way to decide an optimum dosage range of the first drug.

FIG. 13 shows an example of graphs representing the change of efficacy score and toxicity score according to the dosage of a second drug according to an embodiment of the present invention, and shows other way to decide an optimum dosage range of the second drug.

FIG. 14 shows a block diagram of a computing device for executing steps for calculating a drug's efficiency score and the drug's side effect score.

FIG. 15 shows a flow chart of a drug efficiency calculation method according to one embodiment of the present invention.

FIG. 16 shows a flow chart of a drug reactivity calculation method according to one embodiment of the present invention.

### DETAILED DESCRIPTION OF EMBODIMENTS

Embodiments of the present invention will be described with reference to the accompanying drawings. However, the present invention is not limited to the embodiments described herein, and may be implemented in various different forms. The terminology used herein is not for limiting the scope of the present invention but for assisting with an understanding of the embodiments. Furthermore, the singular forms used below include the plural forms as well, unless otherwise indicated.

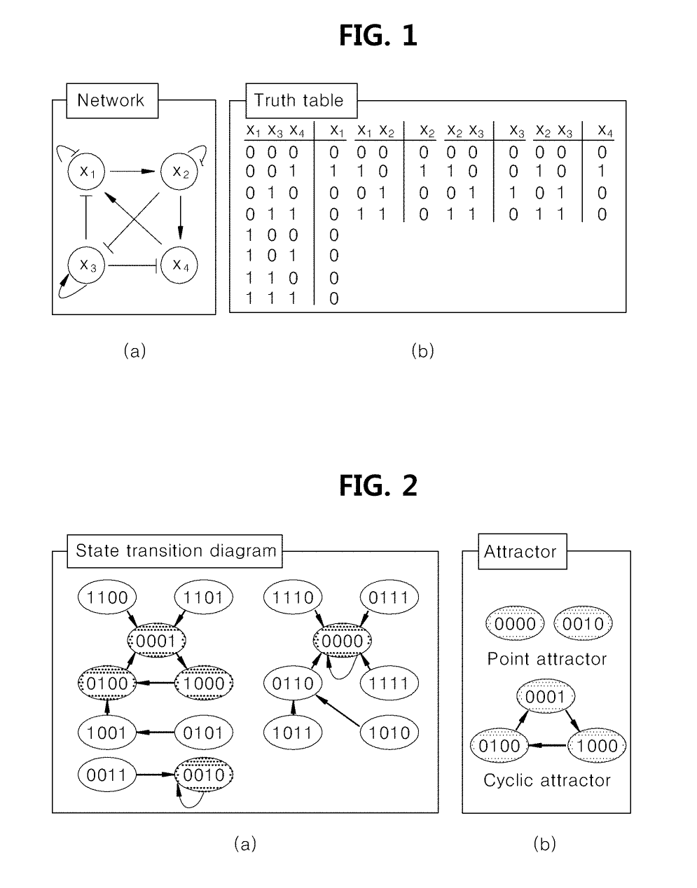

FIG. 1 (a) is a diagram that a bio-molecular network as a network comprising links representing four nodes (x1˜x4) and correlation between them is modeled.

In one embodiment, the value of each node may represent an expression level or an activation degree of a particular function of a bio-molecule corresponding to each node.

In one embodiment, the value of each node may just have binary value of zero (0) or one (1).

In one embodiment, the network shown in FIG. 1 (a) may be a Boolean network.

In one embodiment, when the expression level or the activation degree of a particular function has the value of zero (0) (in other words, when the value of the node corresponding to the particular function is zero (0)), the number of bio-molecules of a first type which activates the particular function in the cell may be regarded as zero (0). And when the expression level or the activation degree of particular function has the value of one (1) (in other words, when the value of the node corresponding to the particular function is one (1)), all of the bio-molecules of the first type in the cell may be regarded as activating the particular function.

In one embodiment, the value of a target node may be related to a probability with which all bio-molecules corresponding to the target node in the cell may activate the target function corresponding to the target node. In other words, the target node may be related to a real number ‘x’ from zero (0) to one (1). Here, when the expression level or the activation degree of the target function has the value ‘x’ (in other words, when the value of the target node corresponding to the target function is ‘x’), it may be regarded that x*100(%) of the bio-molecules corresponding to the target node in the cell show the target function. For example, when the expression level or the activation degree of a target function is related to a value ‘x=1’, it may be regarded as that 100(%) of bio-molecules corresponding to the target node in the cell show the target function. In this case, the target node of the Boolean network shown in FIG. 1 (a) may be related to a probability with which the value of the target node is one (1).

Hereinafter, for convenience of explanation, basically, it is assumed that each node of the Boolean network is designed to have only one of the values zero (0) and one (1).

The arrows in FIG. 1(a) may represent the relationship in which the expression level of the node arranged at the end point of the arrow (=the head portion of the arrow) increases as the expression level of the node arranged at the start point of the arrow increases or the opposite relationship.

The nail shape in FIG. 1(a) may represent the relationship in which the expression level of the node arranged at the end point of the nail shape (=the head portion of the nail) decreases as the expression level of the node arranged at the start point of the nail shape increases or the opposite relationship.

FIG. 1(b) shows a truth table which the values of the four nodes shown in FIG. 1(a) may be have.

FIG. 2(a) is a state transition diagram representing a total of sixteen states which the Boolean network shown in FIG. 1(a) may be have.

FIG. 2(b) is a diagram separately representing two point attractors and one cyclic attractor included in the state transition diagram shown in FIG. 2(a).

The state transition diagram shown in FIG. 2(a) includes a first basin which is a set of states converging on a first point attractor ‘0010’, a second basin which is a set of states converging on a second point attractor ‘0000’, and a third basin which is a set of states converging on a first cyclic attractor [‘0001’, ‘1000’, ‘0100’].

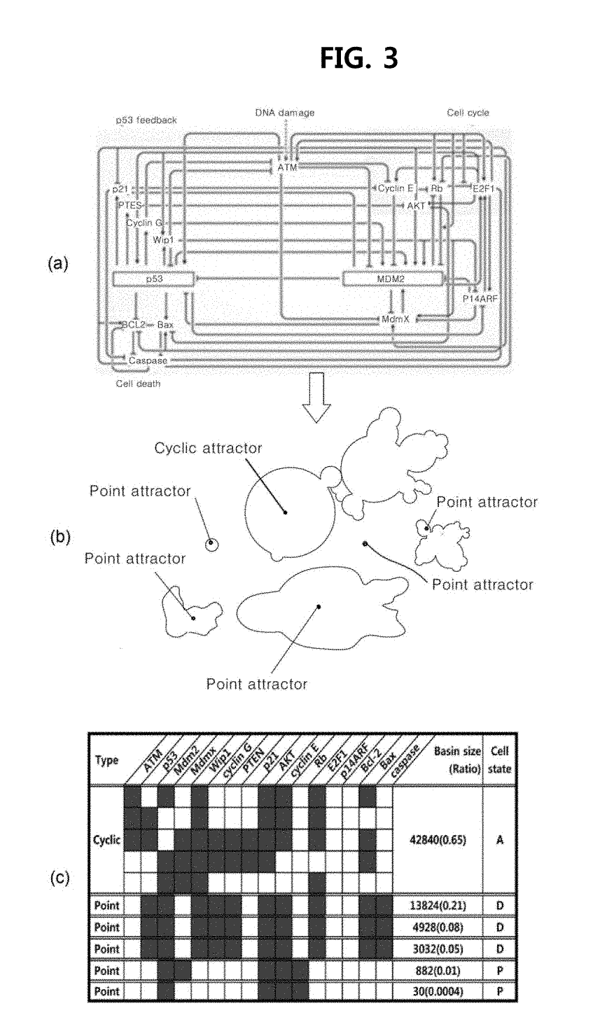

FIG. 3(a) shows a bio-molecular network modeled for a real particular cancer-cell. Each English identifier shown in FIG. 3(a) represents each of the nodes described above, and may represent a gene or a protein. And arrows or nail shapes connecting the nodes represent the link representing interaction between the nodes.

When the bio-molecular network shown in FIG. 3(a) is not a particular cancer-cell but a normal cell, the properties of a particular node or link of the bio-molecular network may be changed. A network representing the normal cell may be called ‘a nominal network’ and a network representing the particular cancer-cell may be called ‘a specific network’. In an embodiment of the present invention, the specific network may be created by changing the nominal network on a basis of the characteristic of the particular cancer-cell. It is understood that in the normal cell and the particular cancer-cell, not only a biochemical reaction modelled on the bio-molecular network shown in FIG. 3(a) but also other biochemical reactions modelled on other bio-molecular networks may occur.

Sixteen nodes are shown in the bio-molecular network shown in the FIG. 3(a). When each node is set to have a binary value, consequently, the bio-molecular network becomes a Boolean network. Here, the number of total states which the Boolean network may have is 2̂16.

FIG. 3(b) is a state transition diagram representing a total of 2̂16 states which the Boolean network shown in FIG. 3(a) may have. The state transition diagram shown in FIG. 3(b) includes five point attractors, one cyclic attractor, and a total of six basins. In the state transition diagram shown in FIG. 3(b), each state may be expressed in a fine point. The areas distinguished by the five closed loop of solid lines are basins of the six attractors. Each basin contains one or a plurality of states of the Boolean network including corresponding attractor.

FIG. 3(c) is a table showing the activity of each node of the Boolean network shown in FIG. 3(a), the basin size (ratio) of the basins shown in FIG. 3(b), and the cell state for each attractor shown in FIG. 3(b), according to the attractors shown in FIG. 3(b).

The first column of the table of FIG. 3(c) indicates the type of the attractors shown in FIG. 3(b). The type is the cyclic type or the point type. The second to 17^{th} column of the table of FIG. 3(c) show each node (ATM, p53, Mdm2, etc., . . . ) the Boolean network shown in FIG. 3(a). The 18^{th} column of the table of FIG. 3(c) shows the basin sizes or the basin ratio of the one cyclic attractor and the five point attractors. The 19^{th} column of the table of FIG. 3(c) shows the cell state for each attractor. In the table, ‘A’, ‘D’, and ‘P’ represents for Arrest, Death, and Proliferation, respectively.

The second to 11^{th} rows of the table of FIG. 3(c) show ten of the possible states of the Boolean network. The second to sixth rows of the table of FIG. 3(c) shows the five cyclic states of the Boolean network for the cyclic attractor shown in FIG. 3(b). Each of the seventh to 11^{th} rows shows the state of the Boolean network for each of the five point attractors shown in FIG. 3(b).

When the Boolean network falls in the cyclic attractor state, the state of the Boolean network repeatedly circulates the five states of the second to sixth rows of the table of FIG. 3(c).

Each cell crossing at the second to 17^{th} column and the second to 11^{th} rows indicates the activity of the corresponding node at the corresponding state of the Boolean network. For example, if a cell is filled in dark texture, the node corresponding to the cell is activated at the corresponding attractor state. In contrast, if a cell is filled in bright texture, the node corresponding to the cell is not activated at the corresponding attractor state.

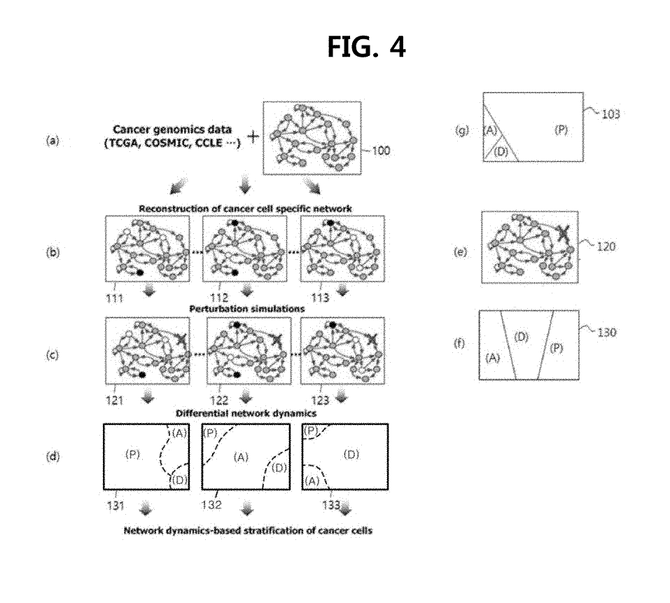

FIG. 4 is a diagram for describing procedure of a drug prediction method through an attractor dynamics analysis of a cancer-cell network provided according to an embodiment of the present invention.

FIG. 4(a) shows the steps of combining cancer genomics data obtained from a database generated from patient groups observed in one or more bio-medical organizations and a cancer-cell line databases created by growing a cancer-cell in a laboratory to a nominal network 100 representing a normal cell.

Nominal network 100 is an example of the above-described bio-molecular network modeled so that each node can have a binary value of 0 or 1.

In a normal cell, not only a biochemical reaction modelled on the nominal network shown in FIG. 4 may occur, but also other biochemical reactions may occur. Thus, with the normal cell, a plurality of different nominal networks may be created. Thus, for the present invention, one among different nominal networks may be selected and used, and the nominal network 100 shown in FIG. 4 is one network among these.

The database generated from patient groups observed in one or more bio-medical organizations may include, for example, TCGA and COSMIC. The TCGA may be a database generated from a first patient group observed in a first bio-medical organization and the COSMIC may be a database generated from a second patient group observed in a second bio-medical organization.

Also, CCLE is an example of the cancer-cell line databases created by growing the cancer-cell in the laboratory.

The cancer genomics data may include a plurality of different cancer-cells.

FIG. 4(b) shows a plurality of different specific networks obtained by mapping the gene mutation information of different cancer-cells to the nominal network 100.

For example, a first specific network 111 may be obtained by mapping gene mutation information of a first cancer-cell to the nominal network 100, a k-th specific network 112 may be obtained by mapping gene mutation information of a k-th cancer-cell to the nominal network 100, and a N-th specific network 113 may be obtained by mapping gene mutation information of a N-th cancer-cell to the nominal network 100.

Because the gene mutation of the first cancer-cell and the gene mutation of the k-th cancer-cell may be different from each other, the first specific network and the k-th specific network may be different from each other.

In the each specific network shown in FIG. 4(b), the each grey node represents a node of a normal state which can have all values of 0 or 1, the each white node represents a first-type limited node which is limited to have only a value representing inactivation of the values of 0 or 1, and the each black node represents a second-type limited node which is limited to have only a value representing activation of the values of 0 or 1.

It may be determined according to the gene mutation information of a cancer-cell mapped to the nominal network 100 which node among the nodes included in the nominal network 100 becomes the first-type limited node or the second-type limited node.

FIG. 4(c) shows a step of changing a specific network into a corresponding specific perturbation network by applying a first perturbation to the specific network. Networks shown in FIG. 4(c) may be named ‘specific perturbation network’ respectively.

Here, the first perturbation may mean an effect of a first drug or a first drug-combination on a cancer-cell. According to the first perturbation, one or more particular nodes are limited to be removed from the bio-molecular network, or each value of one or more particular nodes is limited to have a value of a particular range in the bio-molecular network

For example, a first specific perturbation network 121 may be a network that is generated by applying the first perturbation representing the effect of the first drug or the first drug-combination on a cancer-cell to the first specific network 111.

Likewise, a k-th specific perturbation network 122 may be a network that is generated by applying the first perturbation representing the effect of the first drug or the first drug-combination on the cancer-cell to the k-th specific network 112.

Likewise, a N-th specific perturbation network 123 may be a network that is generated by applying the first perturbation representing the effect of the first drug or the first drug-combination on the cancer-cell to the N-th specific network 113.

The each specific perturbation network is considered a confirmed network which is used to generate a state transition diagram.

FIG. 4(d) shows the perturbation state transition diagrams which are state transition diagrams calculated by the each specific perturbation network.

For example, the first perturbation state transition diagram 131 may be a state transition diagram of the first specific perturbation network 121 modeled when the first drug or the first drug-combination is administered to a first cancer-cell.

And the k-th perturbation state transition diagram 132 may be a state transition diagram of the k-th specific perturbation network 122 modeled when the first drug or the first drug-combination is administered to a k-th cancer-cell.

And the N-th perturbation state transition diagram 133 may be a state transition diagram of the N-th specific perturbation network 123 modeled when the first drug or the first drug-combination is administered to a N-th cancer-cell.

In the example of FIG. 4(d), about each of the first perturbation state transition diagram 131, the k-th perturbation state transition diagram 132, and the N-th perturbation state transition diagram 133, phenotypes with the highest expression probability are cell proliferation (P), cell cycle arrest (A), and cell-death (D) respectively. In other words, when a same drug which is the first drug or the first drug-combination, is administered to the first cancer-cell, the k-th cancer-cell, and the N-th cancer-cell, simulation results may be obtained that the first cancer-cell still proliferates, the k-th cancer-cell ceases to proliferate, and the N-th cancer-cell dies. In other words, effect of a particular drug, such as the first drug or the first drug-combination, may vary from kinds of cancer-cells and how different it is may be known through the computer simulation.

The above-mentioned FIG. 4(c) illustrates networks modified by applying the first drug or the first drug-combination to the corresponding specific network shown in FIG. 4 (b). The above-mentioned FIG. 4 (d) illustrates perturbation state transition diagrams of each of the corresponding networks shown in FIG. 4 (c), respectively. In contrast, by applying a second perturbation different from the first perturbation to the each specific network, the corresponding specific network may be changed. In this way, each specific perturbation network and each perturbation state transition diagram shown in FIG. 4(c) and FIG. 4(d) may be changed.

In other words, information regarding results that different cancer-cells respond to each drug by applying different perturbations representing different drugs to each specific network may be obtained.

FIG. 4(e) shows a step of changing the nominal network 100 by applying the first perturbation to the nominal network 100. In other words, the network shown in FIG. 4(e) may be called ‘a nominal perturbation network 120’. In other words, the nominal perturbation network 120 may be a network created by applying the first perturbation to the nominal network 100, which represents the effect of the first drug or the first drug-combination on the normal cell.

FIG. 4(f) shows ‘a nominal perturbation state transition diagram 130’ which is a state transition diagram calculated by the nominal perturbation network 120. In other words, the nominal perturbation state transition diagram 130 may be a state transition diagram of the nominal perturbation network 120 modelled on the limitation that the first drug or the first drug-combination is administered to the normal cell.

In the embodiment of FIG. 4(f), with regard to the nominal perturbation state transition diagram 130, the phenotype with the highest expression probability is one among cell proliferation (P), cell cycle arrest (A), and cell-death (D). In other words, when the first drug or the first drug-combination is administered to the normal cell, simulation results may be obtained that the normal cell proliferates, or ceases to proliferate, or die. In other words, the effect of a particular drug, for example, the first drug or the first drug-combination on the normal cell may be obtained through computer simulation.

The diagrams of FIG. 4(e) and FIG. 4(f) are obtained by applying the first drug or the first drug-combination to the nominal network 100. Compared to this, by applying a second perturbation different from the first perturbation to the nominal network 100, the nominal perturbation network 120 may be changed. In this way, the nominal perturbation network 120 and nominal perturbation state transition diagram 130 shown in FIG. 4(e) and FIG. 4(f) may be changed.

In other words, information regarding results according to the response of the normal cell on each drug may be obtained by applying different perturbations representing different drugs to the nominal network 100.

Also, ‘a nominal state transition diagram 103’ which is a state transition diagram calculated by the nominal network 100 may be obtained.

FIG. 4(g) shows the nominal state transition diagram 103.

Regarding the nominal state transition diagram 103, the phenotype with the highest expression probability is one of cell proliferation (P), cell cycle arrest (A), and cell-death (D). In other words, simulation results may be obtained that the normal cell to which a drug has not been administered may proliferate, or cease to proliferate, or die.

Now, a method for calculating efficiency of a drug or drugs combination by using perturbation state transition diagrams obtained from the FIG. 4(d) is described. Here, the term ‘efficiency’ is a superordinate concept including an efficacy score and a synergy score.

Also, a method for calculating a side-effect score of a drug or a drug-combination regarding a normal cell is described by using nominal perturbation state transition diagrams obtained from the FIG. 4(f). Here, the side-effect score may be a superordinate concept including a toxicity score and a toxicity synergy score.

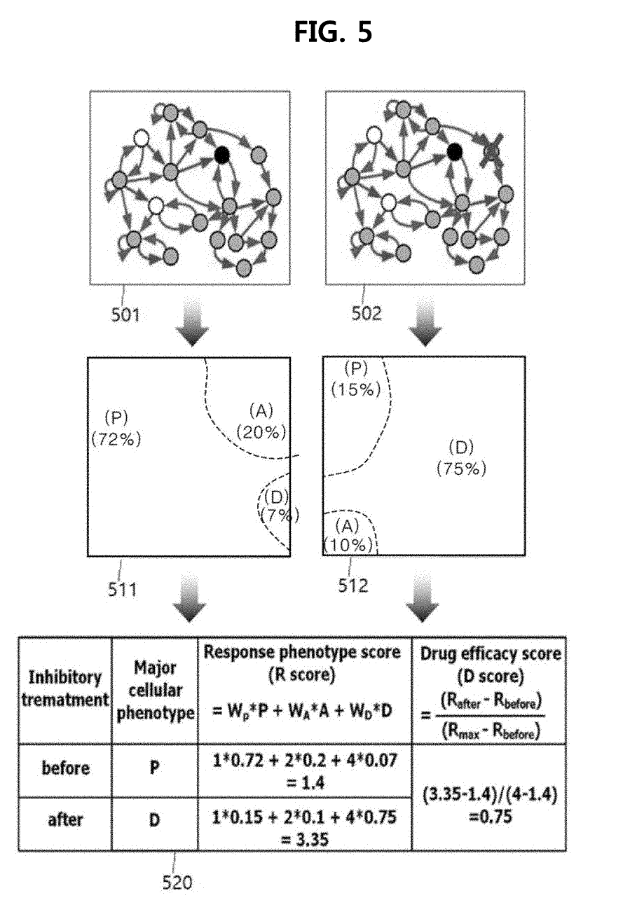

FIG. 5 is a diagram for describing method for calculating the efficacy score of a drug according to an embodiment of the present invention.

In FIG. 5, a second specific perturbation network 502 may be obtained by applying a first perturbation representing an effect of a first drug to a first specific network 501 to which gene mutation information of a first cancer-cell is mapped.

Here, the first state transition diagram 511 may be generated from the first specific network 501 and the second perturbation state transition diagram 512 may be generated from the second specific perturbation network 502.

Now, an efficacy score of the first drug may be calculated by analyzing the first state transition diagram 511 and the second perturbation state transition diagram 512.

The first state transition diagram 511 provides a probability of the phenotype that the first cancer-cell shows when the first drug is not administered to the first cancer-cell. In other words, when the first drug is not administered to the first cancer-cell, probability that the phenotype of the first cancer-cell will become proliferation (P), cycle arrest (A), and death (D) is 72%, 20%, and 7%, respectively.

The second perturbation state transition diagram 512 provides a probability of the phenotype that the first cancer-cell shows when the first drug is administered to the first cancer-cell. In other words, when the first drug is administered to the first cancer-cell, probability that the phenotype of the first cancer-cell will become proliferation (P), cycle arrest (A), and death (D) is 15%, 10%, and 75%, respectively.

Here, the efficacy score of the first drug may be calculated by using method shown in table 520.

The first to fourth columns of the table 520 represent whether an inhibitory treatment has applied or not, the major cellular phenotype, the response phenotype score, and the drug efficacy score, respectively.

The first column of the table 520 represents before the inhibitory treatment is applied, in other words, before the first drug is administered to the first cancer-cell, and the second column of the table 520 represents after the inhibitory treatment is applied, in other words, after the first drug is administered to the first cancer-cell.

In the table 520, since the probability of cell proliferation of the first cancer-cell is 72% before the inhibitory treatment is applied, the major cellular phenotype of the first cancer-cell is ‘proliferation’, but since the probability of cell-death of the first cancer-cell is 75% after the inhibitory treatment is applied, the major cellular phenotype of the first cancer-cell is changed to ‘death’.

The response phenotype score shown in the third column of the table 520, i.e. R score may be defined as the sum of values obtained by multiplying the probabilities which are proportional to the size of each basin according to each state transition diagram, i.e. proliferation probability (PP), cycle arrest probability (PA), second death probability (PD) and the pre-determined weights WP, WA, WD, respectively. In other words, the R score may be defined as FORMULA 1 below.

R score=W_{P}*P_{P}+W_{A}*P_{A}+W_{D}*P_{D} [FORMULA 1]

In FORMULA 1, for example, W_{P}<W_{A}<W_{D} may be set, but not limited to the example. In the example of FIG. 5, W_{P}=1, W_{A}=2, W_{D}=4 are set.

The R score may be calculated for different state transition diagrams, respectively.

In other words, in the example of FIG. 5, it may be understood that the first R score, which is a R score for the first state transition diagram 511, is calculated to be 1.4, and the second R score, which is a R score for the second perturbation state transition diagram 512, is calculated to be 3.35.

Now, in an embodiment of the present invention, The D score, which is the efficacy score for the above-mentioned first drug, may be defined as FORMULA 2.

D score=(R_{after}−R_{before})/(Rₘₐₓ−R_{before}) [FORMULA 2]

Here,

R_{after} is the R score of the cancer-cell after the inhibitory treatment

R_{before} is the R score of the cancer-cell before the inhibitory treatment

Rₘₐₓ is the maximum of the R score which the cancer-cell may be have

In a desirable embodiment, Rₘₐₓ of FORMULA 2 may be equal to W_{D}.

In the example of FIG. 5, it may be understood that the D score of the first drug for the first cancer-cell is calculated to be 0.75.

The D score of case that a second drug different with the first drug or a first drug-combination is administered to the first cancer-cell may also be calculated again.

When the D score regarding a plurality of drugs or drug-combinations about a certain cancel-cell is calculated, a drug or a drug-combination having the largest D score may also be evaluated as the most effective drug or drug-combination in leading to the death of the certain cancer-cell.

Hereinafter, when the first drug or the first drug-combination is administered to the first cancer-cell according to an embodiment of the present invention, method for calculating D score of the first drug or the first drug-combination for the first cancer-cell may include the steps below.

In step S51, a first specific network 501 is generated by mapping gene mutation information of a first cancer-cell to pre-provided nominal network.

In step S52, a second specific perturbation network 502 is generated by applying a first perturbation representing effect of a first drug to the first specific network.

In step S53, a first state transition diagram 511 and a second perturbation state transition diagram 512 are generated from the first specific network 501 and the second specific perturbation network 502 respectively.

In step S54, a first proliferation probability P_{P}_{_}_{before}, a first cycle arrest probability P_{A}_{_}_{before}, and a first death probability P_{D}_{_}_{before} is calculated on a basis of the size of basins which may be obtained in the first state transition diagram 511, a second proliferation probability P_{P}_{_}_{after}, a second cycle arrest probability P_{A}_{_}_{before}, and a second death probability P_{D}_{_}_{before} is calculated on a basis of the size of basins which may be obtained in the second perturbation state transition diagram 512.

In step S55, a first R score R_{before} which is a R score for the first state transition diagram 511 is calculated and a second R score R_{after} which is a R score for the second perturbation state transition diagram 512 is calculated by using the FORMULA 1. And the maximum Rₘₐₓ of the R score according to the FORMULA 1 is calculated.

Here, to calculate the first R score, the first proliferation probability P_{P}_{_}_{before}, the first cycle arrest probability P_{A}_{_}_{before}, and the first death probability P_{D}_{_}_{before} may be applied into P_{P}, P_{A}, P_{D} of FORMULA 1.

And to calculate the second R score, the second proliferation probability P_{P}_{_}_{after}, the second cycle arrest probability P_{A}_{_}_{after}, and the second death probability P_{D}_{_}_{after} may be applied into P_{P}, P_{A}, P_{D} of FORMULA 1.

In step S56, the D score representing the efficacy score of the first drug for the first cancer-cell is calculated by applying the first R score R_{before}, the second R score R_{after}, the maximum Rₘₐₓ of the R score into FORMULA 2.

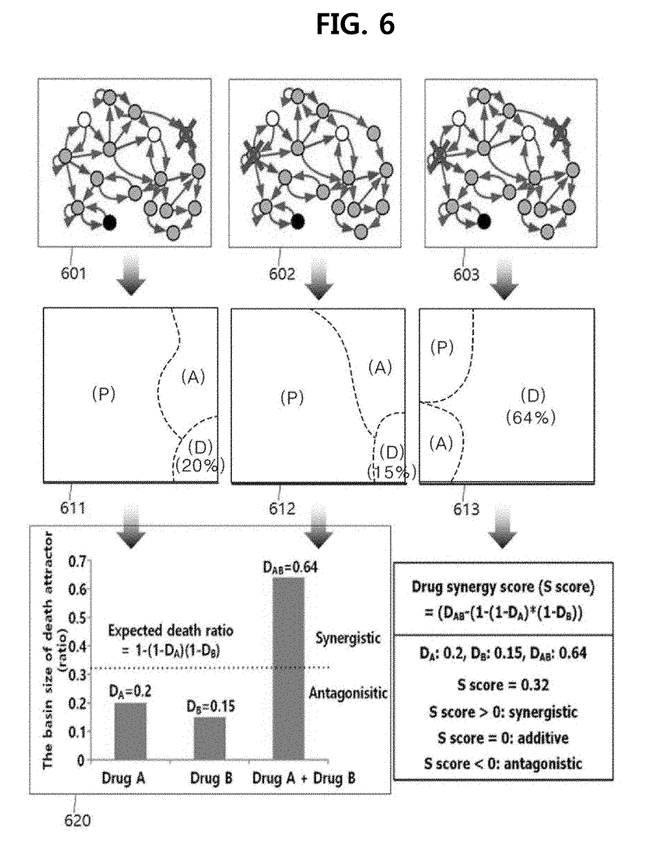

FIG. 6 is a diagram for describing method calculating the synergy score of a drug-combination according to an embodiment of the present invention. Hereinafter, the method is described through the FIG. 6.

The first specific perturbation network 601 may be obtained by applying a first perturbation representing the effect of a drug A to the first specific network to which gene mutation information of a first cancer-cell is mapped.

The second specific perturbation network 602 may be obtained by applying a second perturbation representing the effect of a drug B to the first specific network.

The third specific perturbation network 603 may be obtained by applying a third perturbation representing both the effect of the drug A and the effect of the drug B together to the first specific network.

Here, the first perturbation state transition diagram 611 may be generated from the first specific perturbation network 601, the second perturbation state transition diagram 612 may be generated from the second specific perturbation network 602, and the third perturbation state transition diagram 613 may be generated from the third specific perturbation network 603.

Here, a drug-combination synergy score according to an embodiment of the present invention may be calculated by using the steps below.

In step S61, a specific network is generated by mapping gene mutation information of a first cancer-cell to pre-provided nominal network.

In step S62, a first specific perturbation network 601, a second specific perturbation network 602, and a third specific perturbation network 603 are generated by applying a first perturbation, a second perturbation, and a third perturbation respectively representing the effect of a first drug, a second drug, and a combination of the first drug and the second drug to the specific network.

In step S63, a first perturbation state transition diagram 611, a second perturbation state transition diagram 612, and a third perturbation state transition diagram 613 are generated from the first specific perturbation network 601, the second specific perturbation network 602, and the third specific perturbation network 603, respectively.

In step S64, D-ratios which is a probability with which the first cancer-cell is led to the cell-death state are calculated on the basis of the size of basin representing a cell-death that can be obtained from the first perturbation state transition diagram 611, the second perturbation state transition diagram 612, and the third perturbation state transition diagram 613, respectively.

In step S65, a expected death ratio is calculated by using FORMULA 3.

Expected death ratio=1−(1−D_{A})(1−D_{B}) [FORMULA 3]

Here,

D_{A}, which represents the % size of the basin representing a cell-death in the first specific perturbation network obtained by applying the first perturbation representing the effect of a drug A to the specific network of the first cancer-cell, is a D-ratio by the drug A

D_{B}, which represents the % size of the basin representing a cell-death in the second specific perturbation network obtained by applying the second perturbation representing the effect of a drug B to the specific network of the first cancer-cell, is a D-ratio by the drug B

In step S66, the synergy score S score of the drug-combination is calculated by using FORMULA 4.

S score=D_{AB}−{expected D-ratio}=D_{AB}−{1−(1−D_{A})(1−D_{B})} [FORMULA 4]

Here, D_{AB}, which represents the % size of the basin representing a cell-death in the third specific perturbation network obtained by applying the third perturbation representing the effect that the drug A and the drug B together are administered to the specific network of the first cancer-cell, is a D-ratio by the combination of the drug A and the drug B In the above-mentioned steps S61 to S66, although the drug A and the drug B are assumed to be a single drug, respectively, either the drug A or the drug B may mean a combination-drug in which two or more drugs are already combined. In other word, the synergy score according to the combination of three or more drugs may be calculated.

The graph 620 in the FIG. 6 is for describing the synergy score when the drug A and the drug B are administered together. The value of D-ratio of the drug A (D_{A}) is 0.2, and the value of D-ratio of the drug B (D_{B}) 0.15. Here, ‘Expected D-ratio’ which is just expected when the drug A and the drug B are administered together is 1−(1−0.2)(1−0.15)=0.32. Here, since the D-ratio, D_{AB} is 0.64 when the drug A and the drug B are administered together, the D_{AB} is greater than the ‘Expected D-ratio’ which is 0.32.

If the S score is greater than 0, it may be decided that there is a synergy between the drug A and the drug B (the synergy effect), and if the S score is smaller than 0, it may be decided that there is an opposite relationship between the drug A and the drug B (the resistance effect), and if the S score is 0, it may be decided that there is a simple merger effect.

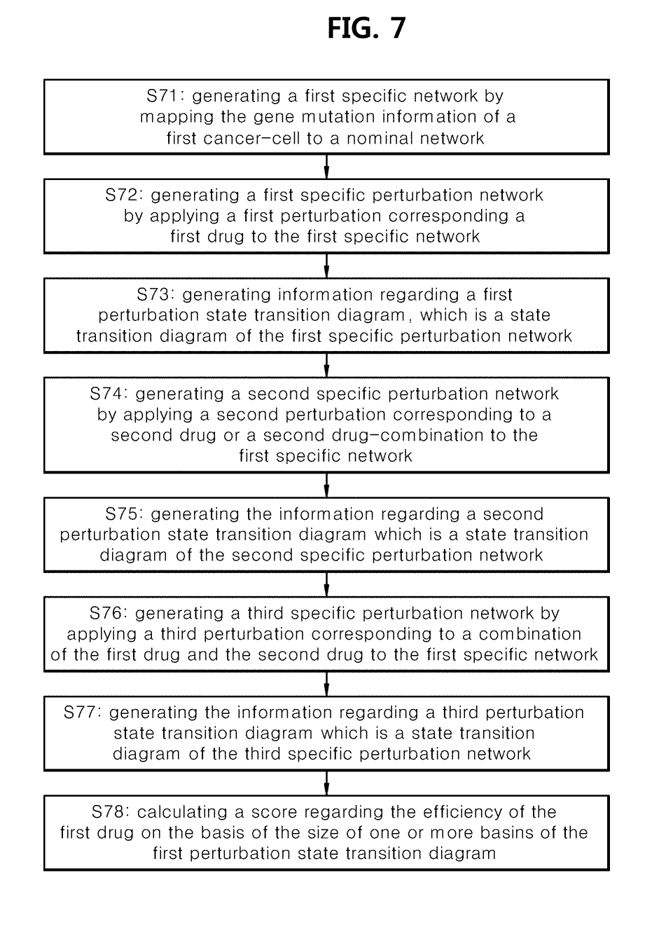

FIG. 7 is a flow chart for describing a drug efficiency calculation method according to an embodiment of the present invention.

In step S71, a first specific network may be generated by mapping the gene mutation information of a first cancer-cell to a nominal network.

In step S72, a first specific perturbation network may be generated by applying a first perturbation corresponding to a first drug to the first specific network.

In step S73, the information regarding a first perturbation state transition diagram, which is a state transition diagram of the first specific perturbation network may be generated.

In step S74, a second specific perturbation network may be generated by applying a second perturbation corresponding to a second drug or a second drug-combination to the first specific network.

In step S75, the information regarding a second perturbation state transition diagram which is a state transition diagram of the second specific perturbation network may be generated.

In step S76, a third specific perturbation network may be generated by applying a third perturbation corresponding to a combination of the first drug and the second drug to the first specific network.

In step S77, the information regarding a third perturbation state transition diagram which is a state transition diagram of the third specific perturbation network may be generated.

In step S78, a score regarding the efficiency of the first drug may be calculated on the basis of the size of one or more basins of the first perturbation state transition diagram.

Here, the score regarding the efficiency may include an efficacy score D score calculated by using the following method.

The efficacy score D score may be calculated by using \textcircled{1} a first R score R_{before} calculated by applying a first proliferation probability P_{P}_{_}_{before}, a first cycle arrest probability P_{A}_{_}_{before}, and a first death probability P_{D}_{_}_{before}, which are respectively proportional to the size of basins representing the cell proliferation, the cell cycle arrest and the cell-death of a first state transition diagram which is the state transition diagram of the first specific network, respectively into P_{P}, P_{A}, P_{D} of FORMULA 1, and \textcircled{2} a second R score R_{after} calculated by applying a second proliferation probability P_{P}_{_}_{after}, a second cycle arrest probability P_{A}_{_}_{after}, and a second death probability P_{D}_{_}_{after}, which are respectively proportional to the size of basins representing the cell proliferation, the cell cycle arrest and the cell-death of the first perturbation state transition diagram, respectively into P_{P}, P_{A}, P_{D} of FORMULA 1.

Here, the score regarding the efficiency may also include a synergy score representing a synergy effect according to the combination of the first drug and the second drug,

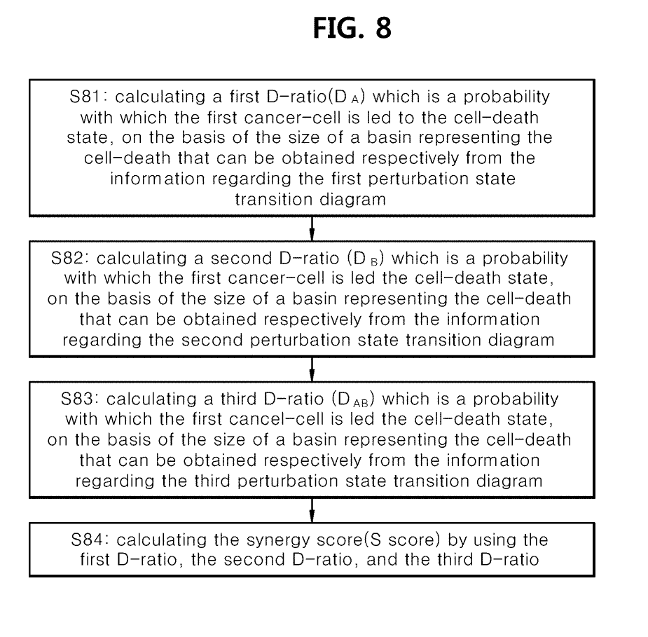

FIG. 8 is a flow chart showing the method for calculating the synergy score shown in FIG. 7.

In step S81, a first D-ratio (D_{A}) which is a probability with which the first cancer-cell is led to the cell-death state, may be calculated on the basis of the size of a basin representing the cell-death that can be obtained respectively from the information regarding the first perturbation state transition diagram.

In step S82, a second D-ratio (D_{B}) which is a probability with which the first cancer-cell is led the cell-death state, may be calculated on the basis of the size of a basin representing the cell-death that can be obtained respectively from the information regarding the second perturbation state transition diagram.

In step S83, a third D-ratio (D_{AB}) which is a probability with which the first cancel-cell is led the cell-death state, may be calculated on the basis of the size of a basin representing the cell-death that can be obtained respectively from the information regarding the third perturbation state transition diagram.

In step S84, the synergy score S score may be calculated by using the first D-ratio, the second D-ratio, and the third D-ratio.

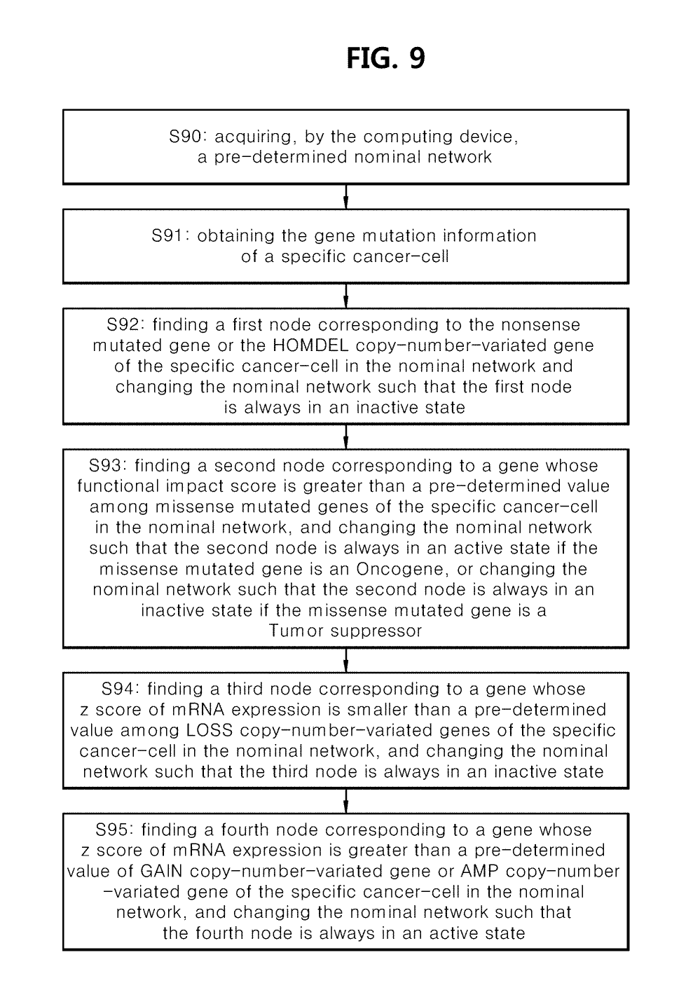

FIG. 9 is a flow chart showing the detailed method for generating the specific network by mapping the gene mutation information of the cancer-cell to the nominal network shown in FIG. 7.

In an embodiment of the present invention, an observable gene mutation (=alteration) may be classified so that it belongs to any one among a nonsense mutation, a missense mutation, a HOMDEL copy-number-variation, a LOSS copy-number-variation, a GAIN copy-number-variation, and an AMP copy-number-variation.

The nonsense mutation may be a change corresponding to the case that some genes in a gene sequence disappear.

The missense mutation may be a change corresponding to the case that some genes in a gene sequence have wrong information when the some genes are compared with genes of a normal state.

To define the HOMDEL copy-number-variation, the LOSS copy-number-variation, the GAIN copy-number-variation, and the AMP copy-number-variation, a gene copy level L3 may be used in case of a normal cell-division.

The LOSS copy-number-variation may be a change corresponding to the case that a gene copy level L2 is lower than the L3 during a cell-division, independently of the information regarding a gene sequence.

The HOMDEL copy-number-variation may be a change corresponding to the case that a gene copy level L1 is lower than the L2 during a cell-division, independently of the information regarding a gene sequence.

The GAIN copy-number-variation may be a change corresponding to the case that a gene copy level L4 is higher than the L3 during a cell-division, independently of the information regarding a gene sequence.

The AMP copy-number-variation may be a change corresponding to the case that a gene copy level L5 is higher than the L4 during a cell-division, independently of the information regarding a gene sequence.

In other words, when CN (gene copy-number-variation type) is defined as a gene copy level, the following relationship is formed.

CN(HOMDEL copy-number-variation)=L1

<CN(LOSS copy-number-variation)=L2

<CN(a normal gene)=L3

<CN(GAIN copy-number-variation)=L4

<CN(AMP copy-number-variation)=L5

According to an embodiment of the present invention, a specific method for generating the specific network by mapping the gene mutation information of the cancer-cell to the nominal network may include the following steps which a computing device performs.

Even if the gene mutation classified as above occurs, it may affect the function of the gene or may not affect the function of the gene.

In step S90, the computing device acquires a pre-determined nominal network. The nominal network may include a plurality of nodes and links representing interrelation between the nodes. At least some among the plurality of nodes may represent genes. Each node of the nominal network may have a active state or an inactive state, stochastically.

In step S91, the computing device may obtain the gene mutation information of a specific cancer-cell. The gene mutation information may include one or more among the nonsense mutation, the missense mutation, the HOMDEL copy-number-variation, the LOSS copy-number-variation, the GAIN copy-number-variation, and the AMP copy-number-variation.

In step S92, the computing device may find a first node corresponding to the nonsense mutated gene or the HOMDEL copy-number-variated gene of the specific cancer-cell in the nominal network and change the nominal network such that the first node is always in an inactive state.

In step S93, the computing device may find a second node corresponding to a gene whose functional impact score is greater than a pre-determined value among missense mutated genes of the specific cancer-cell in the nominal network, and change the nominal network such that the second node is always in an active state if the missense mutated gene is an Oncogene, or change the nominal network such that the second node is always in an inactive state if the missense mutated gene is a Tumor suppressor.

In step S94, the computing device may find a third node corresponding to a gene whose z score of mRNA expression is smaller than a pre-determined value (ex: −2) among LOSS copy-number-variated genes of the specific cancer-cell in the nominal network, and change the nominal network such that the third node is always in an inactive state.

In step S95, the computing device may find a fourth node corresponding to a gene whose z score of mRNA expression is greater than a pre-determined value (ex: +2) of GAIN copy-number-variated gene or AMP copy-number-variated gene of the specific cancer-cell, and change the nominal network such that the fourth node is always in an active state.

Progress performing the step S92 to the step S95 may be regarded as progress mapping the gene mutation information of the specific cancer-cell to the nominal network.

If the step S92 to the step S95 are performed, the specific network may be generated by mapping the gene mutation information of the specific cancer-cell to the nominal network.

Each performance order of the step S92 to the step S95 may be changed each other in freedom.

Meanwhile, the nominal state transition diagram 103 provides a probability of manifestation of the phenotype of the normal cell when the first drug has not been administered to the normal cell. In other words, when the first drug has not been administered, a first set of probabilities of manifestation for proliferation (P), cell cycle arrest (A), and cell-death (D), each of which is a phonotype of the normal cell, can be obtained.

The nominal perturbation state transition diagram 130 provides a probability of manifestation of the phenotype or the normal cell when the first drug is administered to the normal cell. In other words, when the first drug is administered, a second set of probabilities of manifestation for proliferation (P), cell cycle arrest (A), and cell-death (D), each of which is a phonotype of the normal cell, can be obtained

It is easy to understand that the toxicity score of the first drug regarding a normal cell can be calculated by using the first set of probabilities and the second set of probabilities, in a similar way of calculating the D score described in the FORMULA 2. It is easy to understand that the toxicity score is related to a probability with which the normal cell, whose phenotype is desired to be cell proliferation (P) or cell cycle arrest (A), is undesirably led the cell-death state by administered drug.

Also, a toxicity synergy score can be calculated which indicates the difference between a toxicity score observed when a drug A and a drug B are administered together and a sum of a first toxicity observed when the drug A is solely administered solely and a second toxicity observed when the drug B is solely administered. It is easy to understand that the toxicity synergy score can be calculated in a similar way of calculating the S score described in FIG. 6

<A Drug Reactivity Calculation Method>

Hereinafter, a drug reactivity calculation method according to an embodiment of the present invention is described.

The drug reactivity calculation method includes a step of calculating a side-effect score according to a drug administration and a step of calculating an efficiency score according to the drug administration.

In detail, the drug reactivity calculation method may include a drug-reactivity calculating process, which includes:

a step of creating, by a computing device, a first nominal perturbation network by applying a first perturbation to a nominal network modelled on interactions between functions of molecules in a cell, the first perturbation being corresponding to a dosage of a first drug,

a step of creating, by the computing device, information regarding a first nominal state transition diagram which is a state transition diagram of the first nominal network, and information regarding a first nominal perturbation state transition diagram which is a state transition diagram of the first nominal perturbation network,

a step of calculating, by the computing device, a side-effect score of the first drug according to the dosage of the first drug based on the sizes of basins of the first nominal state transition diagram and the sizes of basins of the first nominal perturbation state transition diagram,

a step of creating, by the computing device, a first specific network by mapping gene mutation information of a first cancer-cell to the nominal network,

a step of creating a first specific perturbation network by applying a first perturbation to the first specific network, the first perturbation being corresponding to the dosage of the first drug,

a step of creating, by the computing device, information regarding a first state transition diagram which is a state transition diagram of the first specific network and information regarding a first perturbation state transition diagram which is a state transition diagram of the first specific perturbation network,

a step of calculating, by the computing device, an efficiency score of the first drug according to the dosage of the first drug based on the sizes of basins of the first state transition diagram and the sizes of basins of the first perturbation state transition diagram.

Here, the nominal network may be defined to comprise a plurality of nodes each of which corresponds to the function of the corresponding molecules in the cell and a plurality of links representing the directions of signals delivered between the plurality of nodes and the values of the signals.

For example, when a first protein and a second protein in a cell perform different function, the first protein may be represented by a first node and the second protein may be represented by a second node. Also, for example, when a third protein in the cell performs a first function and a second function, the third protein which performs the first function may be represented by a third node, and the third protein which performs the second function may be represented by a fourth node.

And the directions of signals may be defined by shapes of both end portions of line representing the links. In other words, one end portion of the line may have a nail shape, but the other end portion of the line may have nothing. Here, the directions of the signals may be directed from the said the other end portion to the one end portion. Here a node connected with the above mentioned the other end portion may be called a source node and a node connected with the one end portion may be called a target node.

And the values of the signals may represent a correlation between a value of the source node and a value of the target node. When the shape of the one end portion is an arrow shape, the correlation may be a positive correlation, but when the shape of the one end portion is a nail shape, the correlation may be a negative correlation. The positive correlation may mean that expression of a function represented by the source node promotes expression of a function represented by the target node, and the negative correlation may mean that expression of a function represented by the source node suppresses expression of a function represented by the target node.

Here, in an embodiment, for each of the plurality of nodes, the value of the node is a probability that the function of a molecule in the cell corresponding to the node is activated. In other words, the value of the node may be a value representing a ratio which the function corresponding to the node is activated. Or the value of the node may be a value representing a probability with which all molecules in the cell which may have the function corresponding to the node are activated with the function

Here, for each of the links, when the value of the source node which is a source of the signal on the link of the plurality of nodes is changed, the value of the target node which is a target of the signal on the link is changed according to the value of the signal on the link.

Here, the value of one node of the plurality of nodes is controlled by the first perturbation, or the value of the signal on one link of the plurality of links is controlled by the first perturbation.

Or in another embodiment, the computing device is configured so that the probability is set to only zero (0) or one (1). Here, the computing device is configured so that the value of the signal is set to only a first value or a second value, the first value being a value by which the value of the target node increases when the value of the source node increases, and the second value being a value by which the value of the target node decreases when the value of the source node increases.

In an embodiment, the computing device is configured so that the probability is set to a real number from zero (0) to one (1). Here, the computing device is configured so that the value of the signal output from the source node increases when the value of the source node increases.

Here, in the drug reactivity calculation method, the strength of the first perturbation may be positively correlated with the dosage of the first drug. And the drug reactivity calculation method may further include a data acquisition process acquiring drug-reactivity data having pairs of [toxicity score, efficacy score] according to the dosage of the first drug by conducting the drug-reactivity calculating process a plurality of times with changing the strength of the first perturbation.

Here, the toxicity score may be calculated based on the sizes of the basins of a first nominal state transition diagram which is a state transition diagram of the nominal network, and the sizes of the basins of the first nominal perturbation-state transition diagram.

And the efficacy score may be calculated based on the sizes of the basins of a first state transition diagram which is a state transition diagram of the first specific network, and the sizes of the basins of the first perturbation state transition diagram.

Here, the drug reactivity calculation method may further include an optimal dosage-range determination process calculating the dosage-range of the first drug which makes the toxicity score fall on a first predetermined score range 1210, and the efficacy score fall on a second predetermined score range 1110.

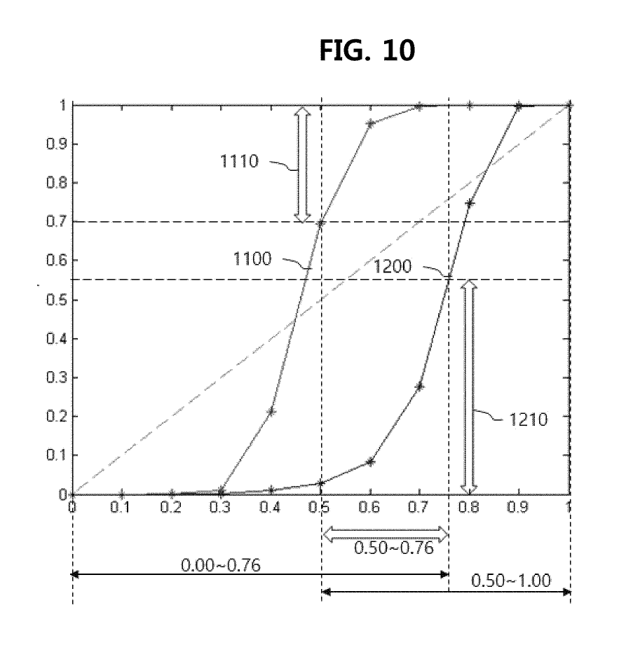

FIG. 10 shows an example of a graph simulating changes of the efficacy score and the toxicity score according to a dosage of a first drug according to an embodiment of the present invention.

In FIG. 10, the horizontal axis represents degrees of suppression that a function of a particular target node or a particular target link affected by a first drug is suppressed according to the dosage of the first drug. For example, points whose degrees of suppression are 1.0, 0.5, and 0.0 represent that function of the particular node or the particular link is suppressed by 100%, 50%, and 0%, respectively. In other words, the function of the particular target node is inhibited by a certain possibility shown in the x-axis of FIG. 10, according to the dosage of the first drug.

Correlation between a dosage of the first drug and the degree of suppression may be presented separately.

In FIG. 10, the vertical axis represents a cell death possibility of a cancer-cell and a cell death possibility of a normal cell.

In FIG. 10, the graph 1100 represents an efficacy score of the first drug for the cancer-cell, and the graph 1200 represents a toxicity score of the first drug for the normal cell. In FIG. 10, data (cross mark) regarding a total of 11 degrees of suppression are presented, and each graph in FIG. 10 represents that linear interpolation is applied between the data.

Here, for example, a dosage of the first drug may be adjusted so that 70%˜100% of cancer-cells are lead to the cell-death state and 0%˜55% of normal cells are lead to the cell-death state. Referring to FIG. 10, in order that 70%˜100% of cancer-cells are lead to the cell-death state, the degree of suppression should be from 0.50 to 1.00, and in order that 0%˜55% of normal cells are lead to the cell-death, the degree of suppression should be from 0.00 to 0.76. Therefore, for example in order that 70%˜100% of cancer-cells are lead to the cell-death state and 0%˜55% of normal cells are lead to the cell-death state, a dosage of the first drug should be decided so that the degree of suppression is from 0.5 to 0.76.

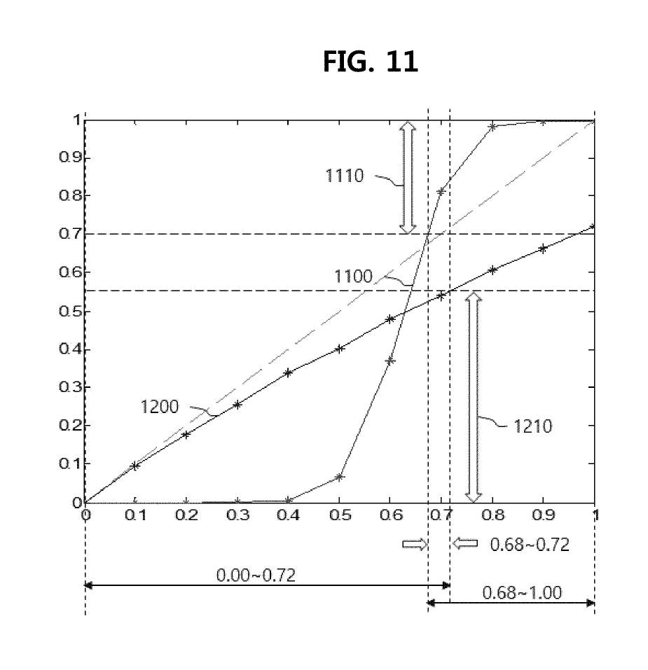

FIG. 11 shows an example of the graph simulating the change of the efficacy score and the toxicity score according to a dosage of a second drug different from the first drug according to an embodiment of the present invention.

The horizontal axis and the vertical axis of FIG. 11 represent same information as the horizontal axis and the vertical axis of FIG. 10.

Correlation between a dosage of the second drug and the degree of suppression may be presented separately.

The graph 1110 of FIG. 11 shows the efficacy score of the second drug for a cancer-cell and the graph 1210 FIG. 11 shows the toxicity score of the second drug for a normal-cell. In FIG. 11, data (cross mark) regarding a total of 11 degrees of suppression are presented, and each graph in FIG. 11 represents that linear interpolation is applied between the data.

Here, along with an explanation regarding FIG. 10, for example a dosage of the second drug may be controlled so that 70%˜100% of cancer-cells are lead to the cell-death state and 0%˜55% of normal cells are lead to the cell-death. Referring to FIG. 11, in order that 70%˜100% of cancer-cells are lead to the cell-death state, the degree of suppression should be from 0.68 to 1.00, and in order that 0%˜55% of normal cells are lead to the cell-death, the degree of suppression should be from 0.68 to 0.72. Therefore, for example in order that 70%˜100% of cancer-cells are lead to the cell-death state and 0%˜55% of normal cells are lead to the cell-death state, a dosage of the second drug should be decided so that the degree of suppression is from 0.68 to 0.72.

When comparing FIG. 10 and FIG. 11, a dosage of the first drug and a dosage of the second drug which should be administered may be different in order that 70%˜100% of cancer-cells are lead to the cell-death state and 0%˜55% of normal cells are lead to the cell-death state.

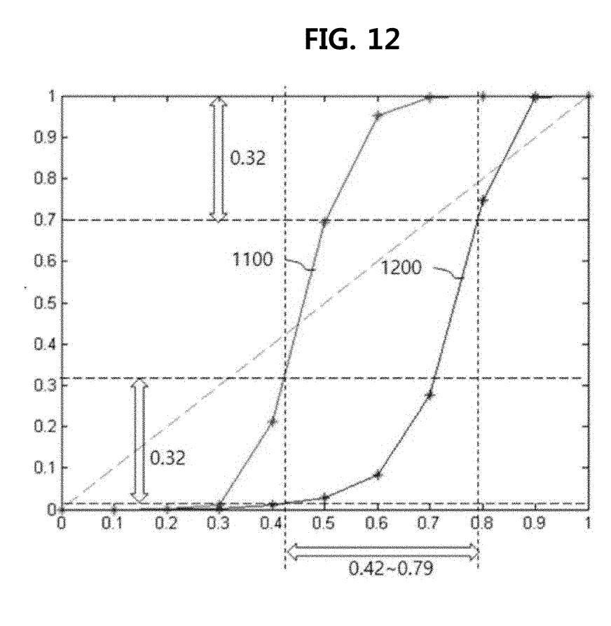

FIG. 12 shows an example of another method to decide a dosage of the first drug according to an embodiment of the present invention.

The graph 1100 and the graph 1200 in FIG. 12 are same with the graph 1100 and the graph 1200 in FIG. 10.

Here, for example, the dosage of the first drug may be adjusted so that the difference value of the possibility that a cancer-cell is lead to the cell-death state and the possibility that a normal cell is lead to the cell-death state is more than 32% when the first drug is administered. Referring to FIG. 12, when the degree of suppression of a target node ranges from 0.42 to 0.79, the difference value of the possibility that a cancer-cell is lead to the cell-death state and the possibility that a normal cell is lead to the cell-death state is more than 32%.

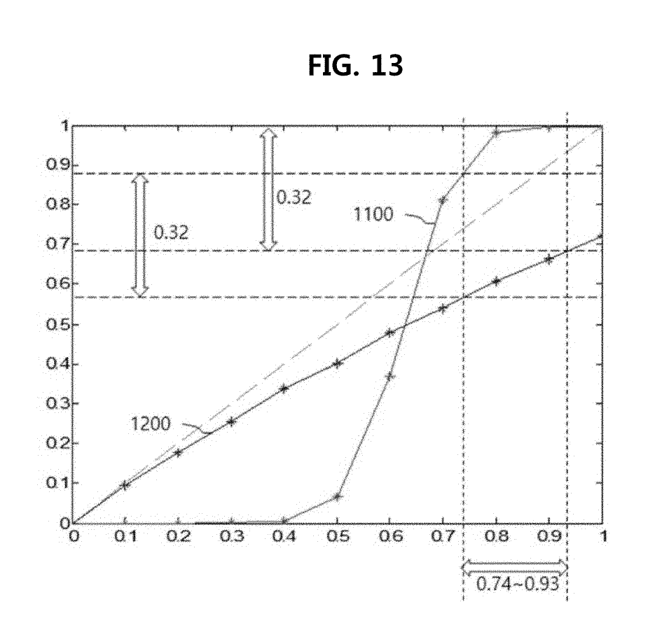

FIG. 13 shows an example of another method to decide a dosage of the second drug according to an embodiment of the present invention.

The graph 1110 and the graph 1210 in FIG. 13 are same with the graph 1110 and the graph 1210 in FIG. 11.

Here, for example, the dosage of the second drug may be adjusted so that the difference value of the possibility that a cancer-cell is lead to the cell-death state and the possibility that a normal cell is lead to the cell-death state is more than 32% when the second drug is administered. Referring to FIG. 13, when the degree of suppression ranges from 0.74 to 0.93, the difference value of the possibility that a cancer-cell is lead to the cell-death state and the possibility that a normal cell is lead to the cell-death state is more than 32%.

When comparing FIG. 12 and FIG. 13, the dosage of the first drug and the dosage of the second drug which should be administered may be different, so that the difference value of the possibility that a cancer-cell is lead to the cell-death state and the possibility that a normal cell is lead to the cell-death state is more than 32%.

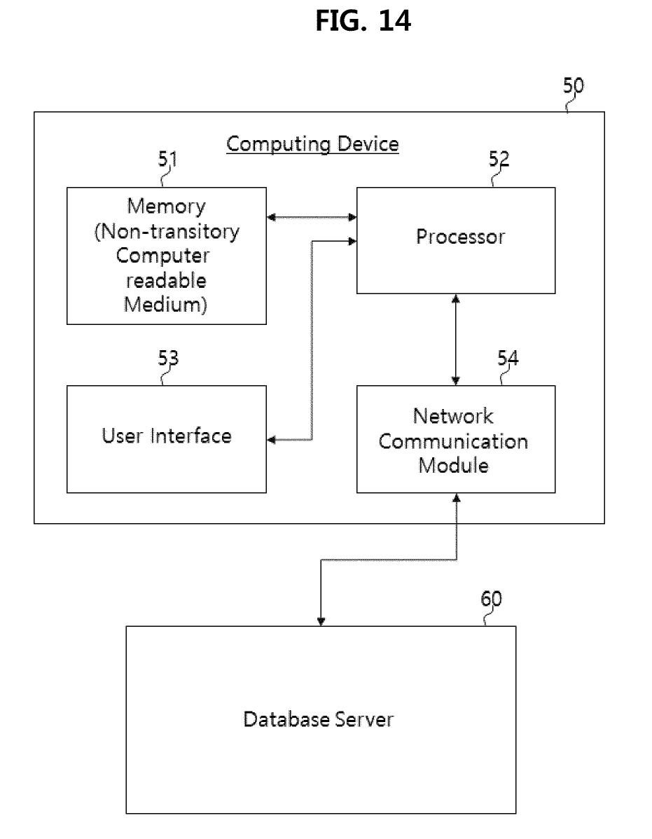

FIG. 14 shows a block diagram of a computing device for executing steps for calculating a drug's efficiency score and the drug's side effect score.

The above mentioned computing device 50 may comprise a memory (a non-transitory computer readable medium) 51, a processor 52, one or more of user interface 53, and a network communication module 54. The above described steps may be conducted by the processor 52 according to instruction codes loaded in the memory 51 with a first data set required for establishing the nominal network, the nominal perturbation network, the nominal state transition diagram, the nominal perturbation state transition diagram, the specific network, the specific perturbation network, the specific state transition diagram, and the specific perturbation state transition diagram in addition with a second data set required for perturbating a target node or a target link of the cellular networks which is related to a drug or a drug-combination. The network communication module 54 may be provided with the first data set and the second data set from a remote database server 60.

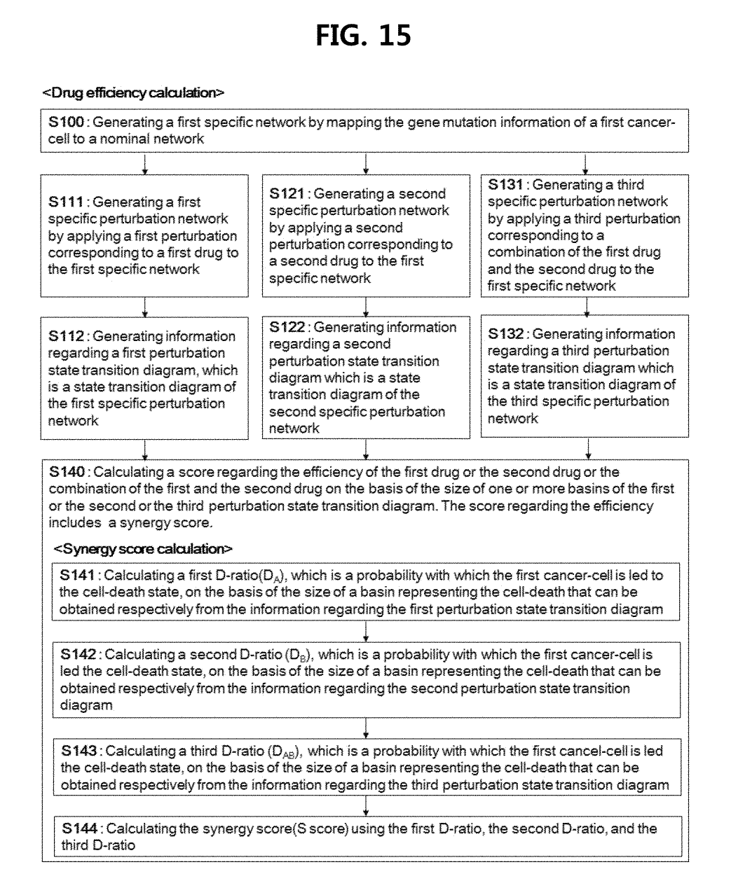

FIG. 15 shows a flow chart of a drug efficiency calculation method according to one embodiment of the present invention. This method may comprise the following steps.

Step S100: Generating a first specific network by mapping the gene mutation information of a first cancer-cell to a nominal network.

Step S111: Generating a first specific perturbation network by applying a first perturbation corresponding to a first drug to the first specific network.

Step S112: Generating information regarding a first perturbation state transition diagram, which is a state transition diagram of the first specific perturbation network.

Step S121: Generating a second specific perturbation network by applying a second perturbation corresponding to a second drug to the first specific network.

Step S122: Generating information regarding a second perturbation state transition diagram which is a state transition diagram of the second specific perturbation network.

Step S131: Generating a third specific perturbation network by applying a third perturbation corresponding to a combination of the first drug and the second drug to the first specific network.

Step S132: Generating information regarding a third perturbation state transition diagram which is a state transition diagram of the third specific perturbation network.

Step S140: Calculating a score regarding the efficiency of the first drug or the second drug or the combination of the first and the second drug on the basis of the size of one or more basins of the first or the second or the third perturbation state transition diagram. The score regarding the efficiency includes a synergy score.

A method for the synergy score may comprise the following steps.

Step S141: Calculating a first D-ratio (D_{A}), which is the probability with which the first cancer-cell is led to the cell-death state, on the basis of the size of a basin representing the cell-death that can be obtained respectively from the information regarding the first perturbation state transition diagram.

Step S142: Calculating a second D-ratio (D_{B}), which is the probability with which the first cancer-cell is led the cell-death state, on the basis of the size of a basin representing the cell-death that can be obtained respectively from the information regarding the second perturbation state transition diagram.

Step S143: Calculating a third D-ratio (D_{AB}), which is the probability with which the first cancel-cell is led the cell-death state, on the basis of the size of a basin representing the cell-death that can be obtained respectively from the information regarding the third perturbation state transition diagram.

Step S144: Calculating the synergy score S score using the first D-ratio, the second D-ratio, and the third D-ratio.

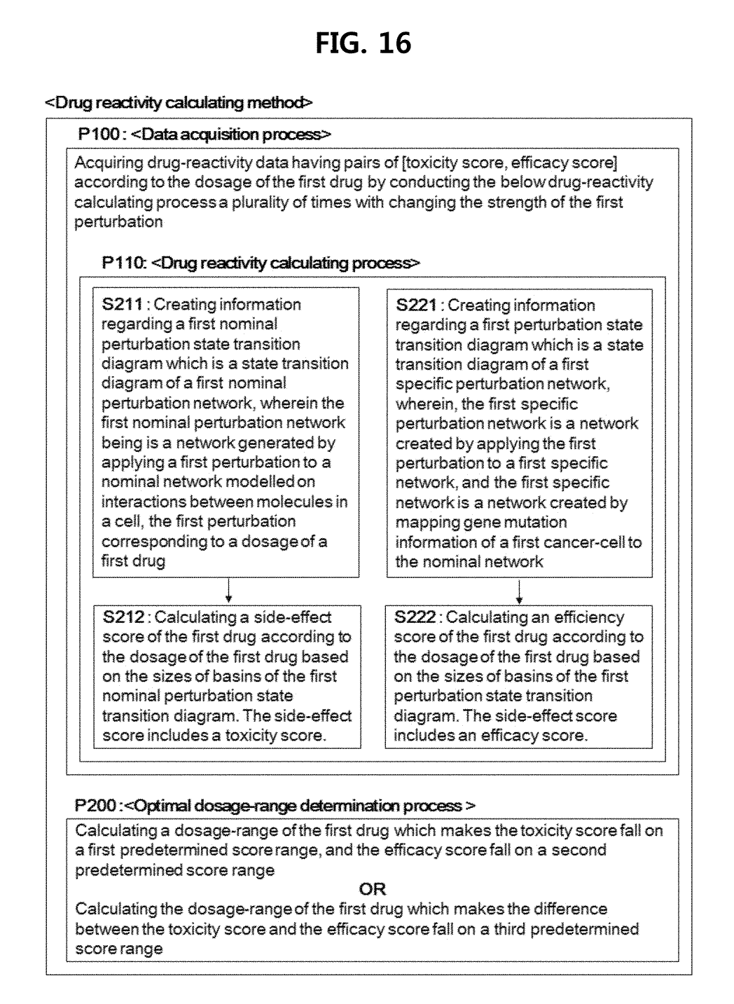

FIG. 16 shows a flow chart of a drug reactivity calculation method according to one embodiment of the present invention. This method may comprise the following processes.

A data acquisition process P100 acquires drug-reactivity data having pairs of [toxicity score, efficacy score] according to the dosage of the first drug by conducting the below drug-reactivity calculating process 110 a plurality of times with changing the strength of the first perturbation.

An optimal dosage-range determination process P200 calculates a dosage-range of the first drug which makes the toxicity score fall on a first predetermined score range 1210, and the efficacy score fall on a second predetermined score range 1110, or calculates the dosage-range of the first drug which makes the difference between the toxicity score and the efficacy score fall on a third predetermined score range. Examples of the third predetermined score range is 0.32˜1.00 shown in FIG. 12 and FIG. 13.

The drug-reactivity calculating process P110 may comprise the following steps.

Step S211: Creating information regarding a first nominal perturbation state transition diagram which is a state transition diagram of a first nominal perturbation network, wherein the first nominal perturbation network being is a network generated by applying a first perturbation to a nominal network modelled on interactions between molecules in a cell, the first perturbation corresponding to a dosage of a first drug

Step S212: Calculating a side-effect score of the first drug according to the dosage of the first drug based on the sizes of basins of the first nominal perturbation state transition diagram. The side-effect score includes a toxicity score.

Step S221: Creating information regarding a first perturbation state transition diagram which is a state transition diagram of a first specific perturbation network, wherein, the first specific perturbation network is a network created by applying the first perturbation to a first specific network, and the first specific network is a network created by mapping gene mutation information of a first cancer-cell to the nominal network

Step S222: Calculating an efficiency score of the first drug according to the dosage of the first drug based on the sizes of basins of the first perturbation state transition diagram. The side-effect score includes an efficacy score.

Those skilled in the art could easily make various alterations or modifications to the above-mentioned embodiments of the present invention without departing the essential characteristics of the present invention. The claims that do not refer to each other may be combined with each other within the scope of understanding of the present disclosure.

What is claimed is:

1. A drug efficiency calculation method, comprising:
generating, by the computing device, information regarding a first perturbation state transition diagram which is a state transition diagram of a first specific perturbation network, wherein, the first specific perturbation network is a network generated by applying a first perturbation corresponding to a first drug to a first specific network, the first specific network being a network generated by mapping gene mutation information of a first cancer-cell to a nominal network; and
calculating, by the computing device, a score regarding the efficiency of the first drug on the basis of the size of one or more basins of the first perturbation state transition diagram.

2. The method of claim 1,
further comprising:
generating, by the computing device, information regarding a second perturbation state transition diagram which is a state transition diagram of a second specific perturbation network, the second specific perturbation network being a network generated by applying a second perturbation corresponding to a second drug to the first specific network; and
generating, by the computing device, information regarding a third perturbation state transition diagram which is a state transition diagram of a third specific perturbation network, the third specific perturbation network being a network generated by applying a third perturbation corresponding to a combination of the first drug and the second drug to the first specific network,
wherein,
the score regarding the efficiency includes a synergy score representing a synergy effect according to the combination of the first drug and the second drug,
the synergy score is calculated by a method including
calculating, by the computing device, a first D-ratio (D_{A}), which is a probability with which the first cancer-cell is led to the cell-death state, on the basis of the size of a basin representing the cell-death that can be obtained respectively from the information regarding the first perturbation state transition diagram;
calculating, by the computing device, a second D-ratio (D_{B}), which is a probability with which the first cancer-cell is led the cell-death state, on the basis of the size of a basin representing the cell-death that can be obtained respectively from the information regarding the second perturbation state transition diagram;
calculating, by the computing device, a third D-ratio (D_{AB}), which is a probability with which the first cancel-cell is led the cell-death state, on the basis of the size of a basin representing the cell-death that can be obtained respectively from the information regarding the third perturbation state transition diagram; and
calculating, by the computing device, the synergy score (S score) using the first D-ratio, the second D-ratio, and the third D-ratio.

3. The method of claim 1, wherein, the score regarding the efficiency includes an efficacy score (D score) which is calculated by using
\textcircled{1} a first R score (R_{before}) calculated by applying a first proliferation probability (P_{P}_{_}_{before}), a first cycle arrest probability (P_{A}_{_}_{before}), and a first death probability (P_{D}_{_}_{before}), which are respectively proportional to the size of basins representing the cell proliferation, the cell cycle arrest and the cell-death of a first state transition diagram which is the state transition diagram of the first specific network, respectively into P_{P}, P_{A}, P_{D} of FORMULA 1, and
\textcircled{2} a second R score (R_{after}) calculated by applying a second proliferation probability (P_{P}_{_}_{after}), a second cycle arrest probability (P_{A}_{_}_{after}), and a second death probability (P_{D}_{_}_{after}), which are respectively proportional to the size of basins representing the cell proliferation, the cell cycle arrest and the cell-death of the first perturbation state transition diagram, respectively into P_{P}, P_{A}, P_{D} of FORMULA 1.
R score=W_{P}*P_{P}+W_{A}*P_{A}+W_{D}*P_{D} [FORMULA 1]
W_{P}, W_{A}, and W_{D} are a pre-determined constant, respectively

4. The method of claim 1, wherein, the nominal network is defined to comprise a plurality of nodes each of which corresponds to the function of the corresponding molecules in the cell and a plurality of links representing the directions of signals delivered between the plurality of nodes and the values of the signals,
for each of the plurality of nodes, the value of the node is 0 (zero) or 1 (one), and
for each of the links, when the value of a source node which is a source of the signal on the link of the plurality of nodes is changed, the value of a target node which is a target of the signal on the link is changed according to the value of the signal on the link,
the value of one node of the plurality of nodes is controlled by the first perturbation, or the value of the signal on one link of the plurality of links is controlled by the first perturbation.

5. A non-transitory computer readable recording medium having recorded thereon program codes to be executed on a computing device to perform the steps of:
generating information regarding a first perturbation state transition diagram which is a state transition diagram of a first specific perturbation network, wherein, the first specific perturbation network is a network generated by applying a first perturbation corresponding to a first drug to a first specific network, the first specific network being a network generated by mapping the gene mutation information of a first cancer-cell to a nominal network; and
calculating a score regarding an efficiency of the first drug on the basis of the size of one or more basins of the first perturbation state transition diagram.

6. A method of calculating reactivity of a drug, comprising:
a drug-reactivity calculating process, which comprises:
creating, by the computing device, information regarding a first nominal perturbation state transition diagram which is a state transition diagram of a first nominal perturbation network, wherein the first nominal perturbation network is a network generated by applying a first perturbation to a nominal network modelled on interactions between molecules in a cell, the first perturbation corresponding to a dosage of a first drug;
calculating, by the computing device, a side-effect score of the first drug according to the dosage of the first drug based on the sizes of basins of the first nominal perturbation state transition diagram;
creating, by the computing device, information regarding a first perturbation state transition diagram which is a state transition diagram of a first specific perturbation network, wherein, the first specific perturbation network is a network created by applying the first perturbation to a first specific network, and the first specific network is a network created by mapping gene mutation information of a first cancer-cell to the nominal network; and
calculating, by the computing device, an efficiency score of the first drug according to the dosage of the first drug based on the sizes of basins of the first perturbation state transition diagram.

7. The method of claim 6, wherein,
the nominal network is defined to comprise a plurality of nodes each of which corresponds to the function of the corresponding molecules in the cell and a plurality of links representing the directions of signals delivered between the plurality of nodes and the values of the signals,
for each of the plurality of nodes, the value of the node is 0 (zero) or 1 (one),
for each of the links, when the value of a source node of the plurality of nodes which is a source of the signal on the link is changed, the value of a target node which is a target of the signal on the link is changed according to the value of the signal on the link, and
the value of selected one node of the plurality of nodes is controlled by the first perturbation, or the value of the signal on selected one link of the plurality of links is controlled by the first perturbation.

8. The method of claim 6, wherein,
the computing device is configured so that the value of the signal is set to only a first value or a second value, the first value being a value which makes the value of the target node increases when the value of the source node increases, and the second value being a value which makes the value of the target node decreases when the value of the source node increases.

9. The method of claim 6, wherein,
the computing device is configured so that,
(1) with a probability of x %, a value of selected one node among the all nodes in a bio-molecular network, which represents the nominal network or the first specific network, is set to 0 (zero), and
(2) with a probability of (100−x) %, a value of the selected one node is set to 0 (zero) or 1 (one), which is determined by the logic ruling the bio-molecular network; and
the computing device is configured so that the value of the signal output from the source node increases when the value of the source node increases.

10. The method of claim 6, wherein,
the strength of the first perturbation is positively correlated with the dosage of the first drug,
the method further comprises a data acquisition process acquiring drug-reactivity data having pairs of [toxicity score, efficacy score] according to the dosage of the first drug by conducting the drug-reactivity calculating process a plurality of times with changing the strength of the first perturbation,
the toxicity score is calculated based on the sizes of the basins of a first nominal state transition diagram which is a state transition diagram of the nominal network, and the sizes of the basins of the first nominal perturbation-state transition diagram, and
the efficacy score is calculated based on the sizes of the basins of a first state transition diagram which is a state transition diagram of the first specific network, and the sizes of the basins of the first perturbation state transition diagram.

11. The method of claim 10, further comprising:
an optimum dosage-range determination process calculating a dosage-range of the first drug which makes the toxicity score fall on a first predetermined score range, and the efficacy score fall on a second predetermined score range.

12. The method of claim 10, further comprising:
an optimum dosage-range determination process calculating a dosage-range of the first drug which makes the difference between the toxicity score and the efficacy score fall on a third predetermined score range.
